(19) United States
(12) Patent Application Publication
(10) Pub. No.: US 2021/0196154 A1
(43) Pub. Date: Jul. 1, 2021

(54) BODY PART CONSISTENCY PATTERN GENERATION USING MOTION ANALYSIS

(71) Applicant: Figur8, Inc., Boston, MA (US)

(72) Inventors: Donna Susan Scarborough, Hingham, MA (US); Shannon Eve Linderman, Cary, NC (US); Nan-Wei Gong, Cambridge, MA (US)

(73) Assignee: Figur8, Inc., Boston, MA (US)

(21) Appl. No.: 17/203,833

(22) Filed: Mar. 17, 2021

Related U.S. Application Data
(63) Continuation-in-part of application No. 16/745,595, filed on Jan. 17, 2020.
Application No. 16/745,595 is a continuation-in-part of application No. 16/529,851, filed on Aug. 2, 2019.
Application No. 16/529,851 is a continuation-in-part of application No. 15/875,311, filed on Jan. 19, 2018, now abandoned.
Application No. 15/875,311 is a continuation-in-part of application No. 15/271,863, filed on Sep. 21, 2016, now abandoned.
(60) Provisional application No. 62/991,113, filed on Mar. 18, 2020.
(60) Provisional application No. 62/821,071, filed on Mar. 20, 2019.
(60) Provisional application No. 62/742,222, filed on Oct. 5, 2018.
(60) Provisional application No. 62/714,241, filed on Aug. 3, 2018.
(60) Provisional application No. 62/513,746, filed on Jun. 1, 2017.
(60) Provisional application No. 62/464,443, filed on Feb. 28, 2017.
(60) Provisional application No. 62/448,525, filed on Jan. 20, 2017.
(60) Provisional application No. 62/221,590, filed on Sep. 21, 2015.

(51) Int. Cl. A61B 5/11; A61B 5/00; A61B 5/107; G16H 40/67; G16H 20/30; G16H 70/60

(52) U.S. Cl. CPC A61B 5/1124; A61B 5/1107; A61B 5/1126; A61B 5/4842; A61B 2562/0219; A61B 5/1073; G16H 40/67; G16H 20/30; G16H 70/60; A61B 5/7282

(57) ABSTRACT

Disclosed embodiments describe techniques for body part biomarker and consistency pattern generation using motion analysis. Sensors are attached to a body part of an individual, where the sensors enable collection of motion data of the body part, and where the sensors include at least one inertial measurement unit (IMU) and at least one sensor determining muscle activation. Data is collected from the sensors, where the sensors provide electrical information based on a microexpression of movement of the body part during a movement performance protocol. Processors are used to analyze the electrical information from the sensors. Biomarker information for the individual is generated using the analyzing of the electrical information from the movement performance protocol. Additional data is collected from a subsequent attaching of sensors to the body part of the individual and the additional data is analyzed. Consistency pattern information is generated from the biomarkers.

### RELATED APPLICATIONS

This application claims the benefit of U.S. provisional patent application “Body Part Consistency Pattern Generation Using Motion Analysis” Ser. No. 62/991,113, filed Mar. 18, 2020.

This application is also a continuation-in-part of U.S. patent application “Movement Biomarker Generation Using Body Part Motion Analysis” Ser. No. 16/745,595, filed Jan. 17, 2020, which claims the benefit of U.S. provisional patent application “Human Body Mounted Sensors with Mapping and Motion Analysis” Ser. No. 62/821,071, filed Mar. 20, 2019.

The U.S. patent application “Movement Biomarker Generation Using Body Part Motion Analysis” Ser. No. 16/745,595, filed Jan. 17, 2020 is also a continuation-in-part of U.S. patent application “Body Part Motion Analysis Using Kinematics” Ser. No. 16/529,851, filed Aug. 2, 2019, which claims the benefit of U.S. provisional patent application “Body Part Motion Analysis Using Kinematics” Ser. No. 62/714,241, filed Aug. 3, 2018, “Wearable Sensors with Ergonomic Assessment Metric Usage” Ser. No. 62/742,222, filed Oct. 5, 2018, and “Human Body Mounted Sensors with Mapping and Motion Analysis” Ser. No. 62/821,071, filed Mar. 20, 2019.

The U.S. patent application “Body Part Motion Analysis Using Kinematics” Ser. No. 16/529,851, filed Aug. 2, 2019 is also a continuation-in-part of U.S. patent application “Body Part Deformation Analysis Using Wearable Body Sensors” Ser. No. 15/875,311, filed Jan. 19, 2018, which claims the benefit of U.S. provisional patent applications “Body Part Deformation Analysis with Wearable Body Sensors” Ser. No. 62/448,525, filed Jan. 20, 2017, “Body Part Deformation Analysis using Wearable Body Sensors” Ser. No. 62/464,443, filed Feb. 28, 2017, and “Body Part Motion Analysis with Wearable Sensors” Ser. No. 62/513,746, filed Jun. 1, 2017.

The U.S. patent application “Body Part Deformation Analysis Using Wearable Body Sensors” Ser. No. 15/875,311, filed Jan. 19, 2018 is also a continuation-in-part of U.S. patent application “Electronic Fabric for Shape Measurement” Ser. No. 15/271,863, filed Sep. 21, 2016, which claims the benefit of U.S. provisional patent application “Electronic Fabric for Shape Measurement” Ser. No. 62/221,590, filed Sep. 21, 2015.

Each of the foregoing applications is hereby incorporated by reference in its entirety.

### FIELD OF ART

This application relates generally to motion analysis, and more particularly to body part consistency pattern generation using motion analysis.

### BACKGROUND

For centuries, people have been fascinated by and argued about objects in motion. Whether the object in motion was an animal such as a human or other mammal transporting themselves by walking, trotting, or galloping; a bird or insect in flight; a tossed object or a fired projectile; or a weaving machine in operation, among many others, people have wanted to understand how those objects moved. The interest in movement likely originated from trying to understand physical phenomena, machine operation, or other movement that could not be detected easily if at all by the human eye. Did a galloping horse have all four hooves off the ground at some point while galloping? What was the number of rotations of a club that was being juggled? What was the wing movement pattern of a hummingbird while hovering? How did a projectile fired from a rifled barrel travel compared to one fired from a smooth barrel? These are just a few of any number of questions about movement that people have wanted to answer. The English photographer, Eadweard Muybridge, was an early developer and adopter of techniques for photography of objects in motion. His work, which included animals moving, horses galloping, men wrestling, and women dancing, showed details about motion that were typically not easily discernable by the naked eye. The work accurately depicted a horse galloping and demonstrated that racehorses did indeed have all four hooves off the ground at some point during their gallop.

Other techniques have been used to study motion. Time-lapse photography is a technique by which a series of photographs is taken over a period of time. The photographs are then strung together or sequenced, and played back at an increased speed. Time-lapse photography is used to show assembly progress along an assembly line, building construction, and other activities that involve movement and assembly of many components, or processes that take a long time to complete. The compression of the elapsed time into a short viewing time enables a person to view the assembly or construction processes in a compressed and logical manner. By contrast, high-speed photography can capture a process or event that occurs far too quickly to be seen by a human observer. A droplet of liquid falling into a pool of the liquid may be visible while falling, but details about the splash caused by the droplet, or the formation of the waves emanating from the droplet landing site, typically are not. Many photographs of the droplet as it falls and splashes into the can be captured within a short period of time. The sequence of many images can be shown over a longer period of time, enabling a viewer to see what actually occurred. The expansion of the short time event into a longer time frame enables the human observer to see details of the event that would be otherwise undetectable.

### SUMMARY

The successful analysis of the motion of a body part is inextricably linked to the accurate measurement of the motion. The analysis of the motion can be used for diagnosing medical conditions or injuries, for measuring efficacy of medical treatment, or for enhancing athletic performance. Techniques for body part movement biomarker and consistency pattern generation using motion analysis are disclosed. At least two sensors, such as inertial measurement units (IMUs) or sensors for determining muscle activation, are applied to a body part of an individual. The electrical characteristics of a given sensor change as the sensor undergoes a change such as movement, stretching, compressing, etc. The electrical characteristics of an IMU change as the IMU accelerates, rotates, or changes position, and the electrical characteristics of the muscle activation sensor change as a muscle flexes or relaxes. The two or more sensors can also include stretch sensors that are based on an electroactive polymer. The electrical characteristics of an electroactive polymer change as the sensor is stretched.

The sensors are attachable to a body part. Tape, wrap, or a garment can be applied to the body part, and the sensors can be attached to the tape, wrap, or garment using hooks. The tape can be a specialized tape such as physical therapy tape, surgical tape, kinesiology therapeutic tape, and so on. One or more strips of tape can be attached to the body part. The one or more strips of tape can be attached in various configurations including a “T”, “W”, “X”, or “Y” configuration. The body part can include one or more of a knee, shoulder, elbow, wrist, hand, finger, thumb, ankle, foot, toe, hip, torso, spine, arm, leg, neck, jaw, head, or back. Data, including changes in electrical information based on microexpression of movement, is collected from the two or more sensors, where the changes in electrical information are caused by motion of the body part or muscle flexion. An at least third sensor can be applied to the body part to determine and analyze motion between symmetrical body parts. The collected electrical information is analyzed to generate consistency pattern information for the individual. The consistency pattern information is used for a clinical evaluation for an individual.

A processor-implemented method for motion analysis is disclosed comprising: attaching two or more sensors to a body part of an individual, wherein the two or more sensors enable collection of motion data of the body part, wherein the two or more sensors include at least one inertial measurement unit (IMU) and at least one sensor determining muscle activation, and wherein the muscle activation comprises muscle deformation timing and muscle deformation displacement; collecting data from the two or more sensors, wherein the two or more sensors provide electrical information based on a microexpression of movement of the body part during a movement performance protocol; analyzing, using one or more processors, the electrical information from the two or more sensors; and generating biomarker information for the individual, using the analyzing of the electrical information from the movement performance protocol. The generated biomarker information is used for a clinical evaluation of the individual where the clinical evaluation includes a degree of injury. The clinical evaluation is monitored over time to produce a healing trajectory. The healing trajectory is used to verify an extent of an injury. In embodiments, the two or more sensors comprise one or more integrated sensors, and the one or more integrated sensors comprise IMUs and muscle activation sensors. In embodiments, the two or more sensors comprise a network of sensors. In embodiments, the two or more sensors capture two or more modalities of body part motion.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for body part biomarker generation using motion analysis.

FIG. 2 is a flow diagram for biomarker usage.

FIG. 3 is an example system for motion evaluation.

FIG. 4 is a flow diagram for calculating a kinetic summation and distribution.

FIG. 5 shows sensor configuration.

FIG. 6 illustrates sensor placement and alternative sensor placement.

FIG. 7A shows shoulder motion.

FIG. 7B shows data collected from shoulders.

FIG. 7C shows sensor position for data collection from the shoulders.

FIG. 8 illustrates a plot of jump data.

FIG. 9 shows a block diagram for a kinematic phase pattern from muscle data.

FIG. 10A shows example lower body sensor locations.

FIG. 10B shows example upper body sensor locations.

FIG. 11 illustrates neck range of motion differences.

FIG. 12 is a system diagram for body part biomarker generation using motion analysis.

### DETAILED DESCRIPTION

Techniques for body part biomarker and consistency pattern generation using motion analysis are disclosed. Two or more wearable sensors can be attached to a body part of an individual. The wearable sensors comprise inertial measurement units (IMUs) for measuring acceleration, rotation, and position of a body part, and muscle activation sensors for determining muscle activation such as microexpressions of body part movement. The wearable sensors can be attached to a fabric which can be attached to a body part. The fabric can include tape, a woven fabric, a knitted fabric, a garment, a wrap, etc. The tape can be a specialized tape such as physical therapy tape, surgical tape, kinesiology therapeutic tape, and so on. The sensors can be used to measure various parameters relating to movement of the body part. The measurement of the body part can be used to perform symmetry evaluation; to evaluate a similar body part; to evaluate symmetrical operation of similar body parts; to perform microexpression movement evaluations; to evaluate angle, force and torque; etc. The body part can include one or more of a knee, shoulder, elbow, wrist, hand, finger, thumb, ankle, foot, toe, hip, torso, spine, arm, leg, neck, jaw, head, or back. The electrical characteristics of the IMU or the sensor for determining muscle activation sensor change as the IMU or the muscle activation sensor moves, stretches, or otherwise reacts to motion of the body part. In embodiments, a stretch sensor may also be used. The electrical information from the sensors can include changes in capacitance, resistance, impedance, inductance, etc. An electrical component coupled to the IMU or muscle activation sensor collects the changes in electrical characteristics produced by the IMU or muscle activation sensor. A communication unit can be coupled to the IMU or muscle activation sensor, and can provide electrical information from the IMU or sensor regarding the changes in electrical characteristics by the IMU or sensor. The electrical information collected from the IMU or muscle activation sensor is analyzed to generate biomarker information for the individual. The biomarker information is used for a clinical evaluation for the individual. The biomarker information is used to generate a consistency pattern.

Traditional IMU-based systems have attempted to infer the “absolute” location of a certain point of interest by integrating the acceleration reading in a 3D space. Thus, IMU systems have been used in various tracking applications such as tracking movement of a body part. However, the accuracy of such an approach is limited by the sampling rate of the IMU and the accuracy of the on-board accelerometer. One problem that is frequently encountered by all of these IMU-based solutions is referred to as drift. Drift is the error (herein location distance) between the actual location of an object versus the location that is calculated/observed by the IMU reading, The drift error results over time from the accumulative error, which is based on the calculation. The approach taken herein includes measuring microexpressions of movement based on determining muscle activation. This approach overcomes the accumulative error. Body movement, or 3D motion of a body part, such as a hand gesture, can be accurately represented in a 3D space over time.

Disclosed techniques address sensing of body part motion and motion analysis for generating body part biomarkers and consistency patterns. In embodiments, tape such as physical therapy tape, therapeutic kinesiology tape, surgical tape, etc. can be applied to a body part. In other embodiments, the body part can be wrapped, placed in a garment, etc. The body part can include one or more of a knee, shoulder, elbow, wrist, hand, finger, thumb, ankle, foot, toe, or hip, or some other body part such as a torso, spine, arm, leg, neck, jaw, head, or back. In embodiments, the tape can be applied to symmetrical body parts such as left shoulder and right shoulder, left hip and right hip, etc. Two or more stretch sensors can be applied to the tape that is applied to a body part. The attaching of the two or more sensors to the tape, wrap, garment, etc., can be accomplished using hooks, a hook and loop technique, fasteners, clips, bands, and so on. The two or more sensors that can be applied can provide electrical information which, when analyzed, can be used to generate biomarker information for an individual. The biomarker information can be used to generate a biomarker for the individual. The biomarker information can be augmented by collecting additional data from a subsequent attachment of an IMU and a muscle activation sensor to the body part. The resulting subsequent biomarker information can be analyzed to generate a consistency pattern. The consistency pattern can be compared to other consistency patterns in a library or analyzed as a change in the individual's biomarkers over time, either of which can enable a clinical evaluation of the individual.

Each muscular-skeletal (MSK) condition has a unique set of biomarkers. Disclosed techniques provide a detailed view of joint motion and muscle function, which can be used to improve accuracy in diagnosis of and treatment for many body-related conditions, such as orthopaedic conditions, degenerative body conditions, accident-related conditions, injury-related conditions, and so on. For example, a biomarker can describe a combination of joint motion with muscle function. This can include peak range of motion of a joint, quality of range of motion of a joint, peak muscle contraction measured in volumetric change, quality of muscle contraction, symmetry of muscle contraction timing in a bilateral moment, stability of motion during an isometric hold pattern, to name just a few.

Microexpressions of movement or motion include the measurable subtleties that a clinically trained eye would not be able to capture. Such microexpressions of motion include the quality of a muscle contraction, timing of activation of a muscle, as well as joint angle movement and velocities. One example of microexpressions can be seen during a test measuring isometric muscle endurance. In this test, an individual is asked to maintain an arm position with shoulders abducted at their sides without movement for up to two minutes. Often a person will shift the arm position to accomplish the task, and the person stops the activity by dropping her arms to her side due to a sense of fatigue. In the case of a motivated individual who wants to work through the sense of fatigue, the shift in arm position is often a subtle movement. The subtle movement can be a slight position change in any direction and is performed to allow other musculature to activate and reduce strain on the primary muscles originally engaged at the start of the task. The clinician observing this task often will not see the subtle change in arm position. This subtle motion can be the way a person completes the task of a timed hold of two minutes, but the person is unknowingly cheating the goal of the test by moving the limb slightly. The techniques disclosed herein enable detection of subtle microexpressions of motion. And in the example just discussed, they can identify both the time in the endurance test when the individual made the subtle position change as well as the magnitude and stability of the position change. Without the ability to capture microexpression of movement, the measurement of isometric endurance is not truly enabled.

Another example of a biomarker that originates from a microexpression is muscle quiver, which can be defined as the shaking of a muscle belly, often as a result of overuse, strain, or direct injury. The ability to capture and measure this subtle motion as a biomarker is a sophisticated digital diagnostic. Muscle quiver differences between an injured person and a non-injured person can be compared. The non-injured biomarker is generally smoother and devoid of rapid slope reversals on a best-fit line of sensor data, such as surface mechanomyography sensor data. The difference between the best-fit data line and the true output data line can be used to generate a numerical quiver score. Other examples of microexpression-based body part motion analysis exist, such as range of motion, symmetry of motion, flexion curve shape, etc.

The inertial measurement unit can include a six-axis or nine-axis IMU. The six-axis IMU can include a gyroscope for three axes, and an accelerometer for three axes. The nine-axis IMU can include a gyroscope for three axes, an accelerometer for three axes, and a magnetometer for an additional three axes. The addition of the magnetometer in the nine-axis IMU can improve accuracy. While the gyroscope and accelerometer can provide information about acceleration and rotation, their accuracy to measure location decreases over time due to drift. The information provided by the magnetometer can provide additional absolute direction sensing. The magnetometer measurements can be used to compensate for the drift over a time interval.

Techniques for motion analysis can be used for body part biomarker generation. The body part biomarker generation can include tracking symmetrical body parts as the body parts are moved based on a movement performance protocol. The movement of the body parts can be related to tracking body part motion, body part diagnosis, body part test, body part therapy, and so on. The body part biomarker generation can be based on acceleration and orientation information from the IMU. The acceleration and orientation information relating to a body part can be collected by a six-axis or a nine-axis inertial measurement unit (IMU). The six-axis IMU can include acceleration and rotation, and the nine-axis IMU can include acceleration, rotation, and absolute direction information. A muscle activation sensor can be used to detect activation of a given muscle. The muscle activation sensor can be based on detectable changes in electrical potential of a muscle. Because muscle activity can be detected and measured in the disclosed techniques, the process can be referred to surface mechanomyography. Depending on the desired biomarker, one or more movement performance protocols can be employed. The movement performance protocols can include both static poses, e.g., hold your arms out straight to the sides for two minutes, or active poses, e.g., do ten squats with your arms out in front of you.

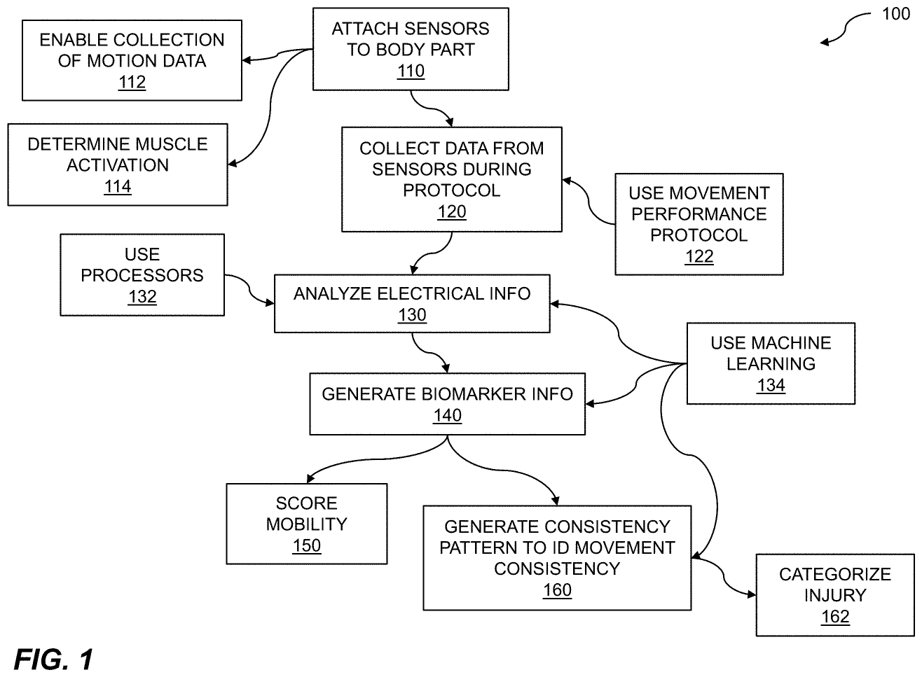

FIG. 1 is a flow diagram for body part biomarker generation using motion analysis. Two or more sensors are used to collect motion data from a body part of an individual. The motion data includes electrical information based on microexpression of movement of the body part. Analysis of the electrical information is used to generate biomarker information for the individual, using the analyzed electrical information. Additional data is collected from a subsequent attaching of the sensors to a body part. The additional data is analyzed to provide additional biomarker information. The additional biomarker information can include longitudinal consistency pattern information for the individual. The consistency pattern information can be used in the context of a clinical evaluation for the individual, a clinical treatment plan for the individual, and so on. Either or both a biomarker generated from the data analysis and an additional biomarker generated from the additional data analysis can comprise the consistency pattern information. Both a biomarker and a consistency pattern can be used for medical diagnosis, treatment of injury, athletic performance improvement, and so on. The flow 100 includes attaching two or more sensors to a body part 110 of an individual. The body part can include a muscle such as a biceps, triceps, or quadriceps muscle; or a joint such as a shoulder, elbow, wrist, hip, knee, or ankle. The attaching can be accomplished using hooks which can attach to tape, a wrap, a garment, etc. The tape can include physical therapy tape and kinesiology therapeutic tape, a woven material, etc. The two or more sensors enable collection of motion data 112 of the body part. The two or more sensors can be the same type of sensor or different types of sensors. In embodiments, the two or more sensors can include at least one inertial measurement unit (IMU) and at least one sensor determining muscle activation 114. An IMU can include a variety of measurement components such as one or more of an accelerometer, a gyroscope, and a magnetometer. The IMU can measure acceleration, rotation, position, and so on. A sensor for determining muscle activation can measure electric potential for a given muscle and can output electrical information. The electrical information can be based on electrical characteristics of the sensor that can be changed based on muscle activation. The electrical characteristics that can be changed can include resistance, capacitance, inductance, etc. Other types of sensors can also be used, such as stretch sensors based on electroactive polymers.

The flow 100 includes collecting data from the two or more sensors 120, where the two or more sensors provide electrical information based on a microexpression of movement of the body part during a movement performance protocol. A movement performance protocol can include task performance such as sitting or standing; performing an operation such as reaching forward, up, down, or to the side; performing a squat; holding a pose; and the like. In embodiments, the electrical information that is provided by the sensors originates from detecting mechanical motion. The electrical information that is collected can be based on a DC signal, an AC signal, pulses, and so on. The electrical information can include values related to resistance, capacitance, inductance, etc. In embodiments, the electrical information that is provided can include surface mechanomyography information. The electrical information that is collected from the two or more sensors can be held or stored within the two or more sensors, transmitted to a receiving component, etc. Transmitting of electrical information can be accomplished using one or more wireless techniques including Wi-Fi, Bluetooth™, Zigbee™ near-field communication (NFC), and the like. Transmitting of the electrical information, when included, can provide continuous transmission, burst transmission, intermittent transmission, etc. The flow 100 can further include analyzing the task performance 122 to identify movement dysfunction. The movement dysfunction can include asymmetrical movement between symmetrical joints such as shoulders. In embodiments, the movement dysfunction that is identified can enable determination of typical injury impairment. Typical injury impairment can include reduced range of motion due to a wrist or ankle sprain, a neck strain, and the like. Typical injury impairment can further include tendonitis.

The flow 100 includes analyzing the electrical information from the two or more sensors 130. The analyzing can include determining motion of a joint such as an ankle, knee, hip, wrist, elbow, or shoulder. The analyzing can be accomplished using one or more processors 132. The analyzing can include determining a range of motion of a joint such as a knee or a shoulder. The analyzing can include comparing symmetrical joints. The comparing symmetrical joints can include comparing ranges of motion of a left knee and a right knee, the relative motions of a left shoulder and a right shoulder, and so on. The analysis can include determining an objective measurement of scapular movement. The analyzing can include kinetic phases. In embodiments, the one or more processors on which the analysis of the electrical information from the two or more sensors is performed can be coupled to the two or more sensors or can be performed beyond the two or more sensors. The one or more processors can include a smart device, a smartphone, PDA, laptop, a local server or blade server, a remote server, a mesh server, a cloud-based server, or a service such as computing as a service (CaaS), etc. The one or more processors can use machine learning 134 to identify electrical information, biomarker information, and consistency pattern information by training the machine using pre-identified examples of desired information. In embodiments, the electrical information from the two or more servers can be analyzed on a tablet. In other embodiments, the two or more sensors comprise one or more integrated sensors. For example, an integrated sensor may include a sensor to detect muscle activation and an IMU to detect muscle movement based on spatial positioning, acceleration, velocity, and so on. In embodiments, the one or more integrated sensors comprise stretch sensors. In further embodiments, the two or more sensors comprise a network of sensors. The network of sensors can be attached to a single body for multiple measurements of complex movement of that body or body part. The network of sensors can be attached to more than one body. For example, several bodies can have two or more sensors attached to the same body part on each body. Comparative muscle movement of each of the bodies is thereby enabled. In embodiments, the two or more sensors capture two or more modalities of body part motion. The modalities can include muscle activation, or contraction, and muscle movement in three dimensions. The modalities can be assessed using a stretch sensor and an IMU, although other sensors for capturing modalities are possible.

The flow 100 includes generating biomarker information 140 for the individual. The generating is accomplished using the analyzing of the electrical information from the movement performance protocol. Biomarker information can be used to create a biomarker, which is a digital representation of a body's state, and in this context, a body's state with respect to movement or motion. The flow 100 includes scoring mobility 150 of the individual, based on the biomarker information. Scoring mobility can be based on a value, a percentage, a range of values, a qualitative assessment, and so on. The scoring mobility can be used to categorize an injury, as described below. The flow 100 includes generating the consistency pattern information to identify movement pattern consistency 160. The movement pattern consistency can be in reference to consistency of movement of a joint, consistency of movement between a pair of joints, and so on. The movement pattern consistency can be in reference to muscle activation such as a left quadriceps, consistency of muscle activations between left quadriceps and right quadriceps, and so on. A movement consistency pattern can be understood as a degree of replication of each piece or segment of a movement. In a usage example, a movement consistency pattern can be applied to left and right shoulder movement commensurate with a forward reach of left and right arms. Movement consistency patterns can be based on collecting and analyzing electrical data associated a movement performance protocol. The electrical data can be associated with movement timing, peak angular velocities, position of left and right shoulders, and so on. A consistency pattern can be generated with respect to an individual's own movement, with respect to an individual's own movement over time (longitudinal analysis), or with respect to a library of others' consistency patterns. Discussed below, the consistency pattern information can be used for a clinical evaluation for the individual. In embodiments, the movement pattern consistency can be used to detect an injury of the individual. Detection of an injury can be based on restricted motion, abnormal motion, lack of motion, and the like. In the flow 100, the detection of an injury can further include categorizing the injury 162 of the individual based on the movement pattern consistency. The categorizing the injury can include determining a degree of injury such as mild or severe; recommending that the injury be treated by surgery or physical therapy; etc.

Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors.

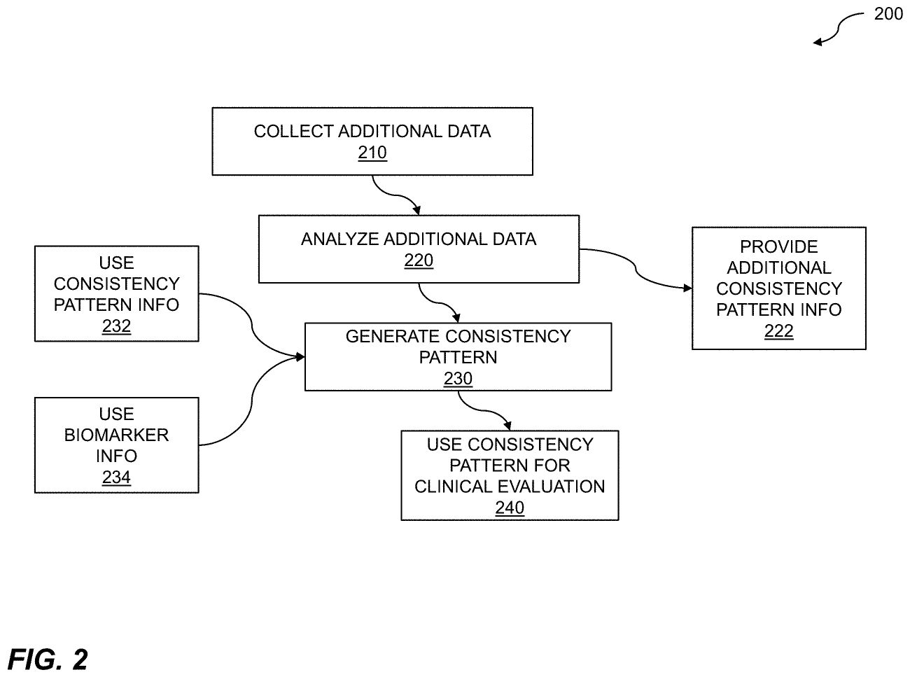

FIG. 2 is a flow diagram for consistency pattern usage. Discussed throughout, consistency pattern information can be generated based on analyzing sensor information collected as an individual performs a movement protocol. The consistency pattern can be used to determine motion symmetry between joints such as shoulders of a human body. Motion analysis is used for body part consistency pattern generation. Sensors are attached to a body part of an individual, where the sensors enable collection of motion data of the body part. The sensors include at least one inertial measurement unit (IMU) and at least one sensor determining muscle activation. Data is collected from the sensors, where the sensors provide electrical information based on a microexpression of movement of the body part during a movement performance protocol. Processors are used to analyze the electrical information from the sensors, and biomarkers and/or consistency pattern information for the individual is generated.

The flow 200 includes collecting additional data 210 from a subsequent attaching of two or more sensors to the body part of the individual. Data can be collected over periods of time for a variety of purposes including gauging deterioration of a joint or joints, progression of a disease, recovery from an injury or surgery, and so on. The data that can be collected can include longitudinal data. The collecting additional data can be used to compare data collected from the individual with data collected from one or more other individuals. The additional data can comprise biomarker information and biomarkers. The flow 200 includes analyzing the additional data 220 to provide additional information. The additional information can include further sensor data based on microexpressions of movements of the body part. The analyzing can include analyzing further electrical information from the sensors. The electrical information can be based on a voltage or current; a frequency; a resistance, capacitance, or inductance; and the like. In the flow 200, the analyzing additional data can provide additional consistency pattern information 222.

The flow 200 further includes generating a consistency pattern 230. The consistency pattern can be related to the first consistency pattern, can be a continuation of the first consistency pattern, can be separate from the first consistency pattern, and so on. In the flow 200, the generating the consistency pattern uses the consistency pattern information 232 generated previously and the additional consistency pattern information. In the flow 200, the generating the consistency pattern uses biomarker information 234 generated previously for the individual. The flow 200 includes using the consistency pattern for a clinical evaluation 240 for the individual. The clinical evaluation of the individual can include determining an extent of an injury, progress of recovery, efficacy of a rehabilitation protocol such as physical therapy, and so on. In embodiments, the clinical evaluation can enable determination of malingering. The determination of malingering can be used to evaluate whether pain reported by an individual is due to a physical injury or is psychosomatic. In other embodiments, the clinical evaluation can enable classification of a degree of an injury. The injury that can be classified can include injury to a limb, a muscle, a joint, and so on. In embodiments, the injury can include a neck strain. The classifying the injury can include determining an extent or degree of the injury. The classifying the injury such as neck strain can include a degree of mild or severe. Discussed throughout, the clinical evaluation can be used for further purposes. In embodiments, the clinical evaluation can be monitored over time to produce a healing trajectory. Other uses of the clinical evaluation can include comparing the healing trajectory of the individual to a library of healing trajectories. Various steps in the flow 200 may be changed in order, repeated, omitted, or the like without departing from the disclosed concepts. Various embodiments of the flow 200 can be included in a computer program product embodied in a non-transitory computer readable medium that includes code executable by one or more processors.

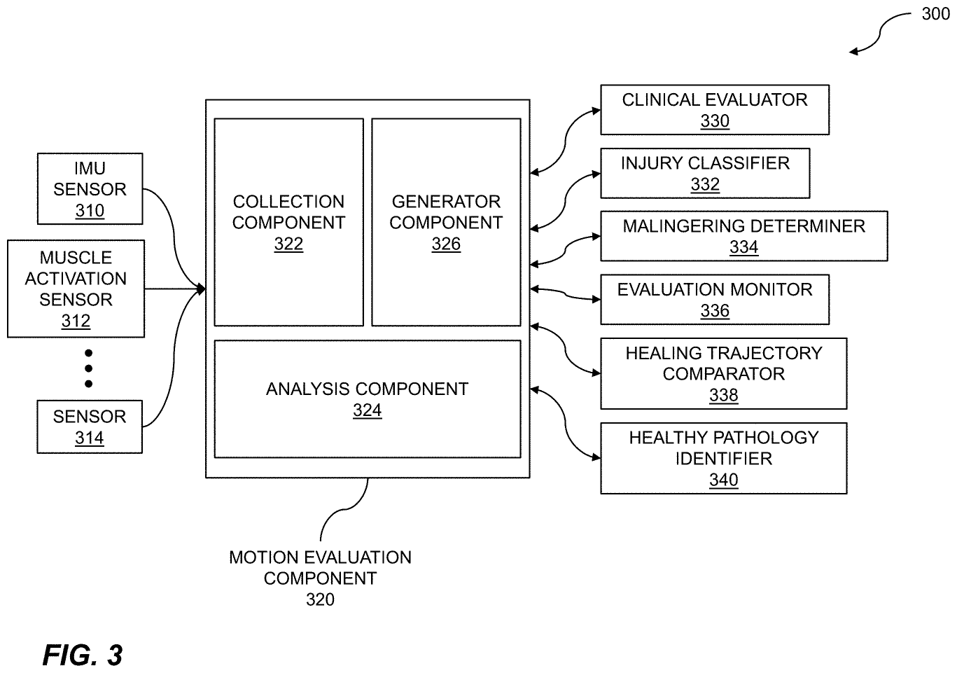

FIG. 3 is an example system for motion evaluation. The motion evaluation can be based on a consistency pattern, on biomarkers, and on other criteria or parameters. A consistency pattern can be based on a consistency of movement of a given body part such as a joint, a limb, or a muscle; on symmetric movement of joints such as left and right shoulders; and so on. The consistency pattern can be used for a variety of purposes such as performing a clinical evaluation; determining a clinical treatment plan; evaluating a degree of injury, recovery from surgery, or the possibility of malingering; and so on. Markers such as biomarkers can include movement biomarkers which can be generated. The consistency pattern information, the movement biomarkers, etc., can be generated over a period of time. Motion analysis is used for body part consistency pattern generation. Sensors are attached to a body part of an individual, where the sensors enable collection of motion data of the body part, and where the motion data includes at least one inertial measurement unit (IMU) and at least one sensor determining muscle activation. Data is collected from the sensors, where the sensors provide electrical information based on a microexpression of movement of the body part during a movement performance protocol. Processors are used to analyze the electrical information from the two or more sensors, and consistency pattern information is generated. The consistency pattern information that is generated can be based on one or more biomarkers. Consistency pattern information for an individual is generated using the electrical information that was analyzed.

An example system for motion evaluation 300 is shown. The system can include two or more sensors, where the two or more sensors can be attached to a body part of an individual. The sensors can include an IMU sensor 310, a muscle activation sensor 312, another sensor 314, and so on. While three sensors are shown, other numbers of sensors can be used. The body part can include a muscle of a torso such as a pectoral; a deltoid; a muscle of a limb such as a biceps, triceps, or quadriceps muscle; and so on. The body part can include a limb such as an arm or a leg. The body part can include a joint such as a shoulder, elbow, wrist, hip, knee, or ankle. The attaching of the sensors can be accomplished using hooks, where the hooks can attach to tape, a wrap, a garment, etc. The two or more sensors enable collection of data such as motion data of the body part. The sensors can be similar types of sensors or different types of sensors. The sensors can include sensors for determining muscle activation. The sensors for detecting muscle activation can include a load cell, a torque sensor, and the like. In embodiments, the sensor can include an electroactive polymer. An electroactive polymer can include a polymer that changes size or shape when activated by an electrical signal or field. The electrical characteristics of the electroactive polymer change based on stretching or deformation. The electrical characteristics that can change can include resistance, capacitance, inductance, etc. Other types of sensors can also be used. The sensors can include inertial measurement units (IMUs). An IMU can include one or more of an accelerometer, a gyroscope, and a magnetometer. The IMU can measure acceleration, rotation, position, and so on. The use of sensors further to the at least two sensors can confirm data collected from the first two sensors using a majority vote or other technique, can provide additional information, etc. An additional sensor can enable body part symmetry analysis such as range of motion, stretch, and so on.

The system 300 includes a motion evaluation component 320. The motion evaluation component can include one or more processors. The one or more processors can include a smart device, a smartphone, PDA, a tablet, a laptop, a local server or blade server, a remote server, a mesh server, a cloud-based server or service such as computing as a service (CaaS), and the like. The motion evaluation component can include a collection component 322. The collection component can collect data from sensors such as sensor 310, sensor 312, sensor 314, and so on. The data that is collected can include electrical data, where the electrical data can include a voltage, a current, a frequency, an offset, a phase, etc. The data such as electrical data can be collected continuously, periodically, occasionally, and so on. The data can be stored within the motion evaluation component; within a device such as a tablet, smartphone, or PDA; on a local server or a remote server; and the like. The motion evaluation component can include an analysis component 324. The analysis component can use the one or more processors to analyze the electrical information collected from the sensors. The analyzing can include determining motion of a muscle such as a deltoid, biceps, quadriceps, and so on. The analyzing can include determining motion of a joint such as an ankle, knee, hip, wrist, elbow, or shoulder. The analyzing can include determining a range of motion of a joint such as a knee or a shoulder. The analyzing can include comparing motion of symmetrical joints such as knees or shoulders. The analysis can include determining an objective measurement of scapular movement. The analyzing can include kinetic phases. In embodiments, analysis of the electrical information by the analysis component can be performed on a smart device, a smartphone, PDA, tablet, laptop, a local or remote server, a cloud-based server, etc. The motion evaluation component can include a generator component 326. The generator component can generate biomarker and/or consistency pattern information for the individual by using the analyzed electrical information from the sensors. Discussed throughout, biomarker information can include a measurable index of body part movement ability, and consistency pattern information can include a measurable motion pattern associated with a body or body part. The biomarker or consistency pattern information can be used to indicate a biological condition or quality such as healthy or injured, or a biological state such as in motion or at rest, etc. The biomarker or consistency pattern information can be generated to determine a degree of injury, to form a recommendation or series of recommendations for treatment or therapy, to measure progress of healing, and so on.

The system 300 includes one or more components that can use one or more biomarkers or one or more consistency patterns for a variety of applications or purposes. In embodiments, the system 300 includes a clinical evaluator 330. The clinical evaluator can use a consistency pattern within a clinical evaluation of an individual. The consistency pattern can be used to identify asymmetry within motion, range of motion, and so on, between symmetrical joints. The symmetrical joints can include ankles, knees, hips, wrists, elbows, or shoulders. In embodiments, consistency pattern information can be used to perform a clinical evaluation such as determining scapular dyskinesia. The system 300 includes an injury classifier 332. The injury classifier can enable classification of a degree of an injury. The degree of injury can include mild, moderate, severe, catastrophic, etc. In embodiments, the injury that is classified can include a neck strain. In other embodiments, the system 300 includes a malingering determiner 334. The malingering determiner can be used as part of a clinical evaluation, where the clinical evaluation determines that a patient is indeed not injured or perhaps only mildly injured. For example, the malingering can be a part of an exaggerated or falsified claim of whiplash, which is made evident through analysis of the consistency pattern evaluated during one or more prescribed movements of an individual making the claim.

In embodiments, the system 300 includes an evaluation monitor 336. The evaluation monitor can be used to monitor a clinical evaluation over time. The monitoring of the clinical evaluation over time can be used to determine whether a particular therapy is effective, whether an injury is healing, and so on. The system 300 includes a healing trajectory comparator 338. The healing trajectory comparator can be used to verify an extent of an injury, to determine whether healing from the injury or from surgery is progressing, and so on. The healing trajectory, which can be produced by monitoring a clinical evaluation over time, can be compared to a library of healing trajectories. The library of healing trajectories can include trajectories for normal healing, typical healing, etc. In embodiments, the healing trajectory can be used to verify injury extent. The system 300 includes a healthy pathology identifier 340. A pathology can be healthy, unhealthy, injured, and so on. In embodiments, the clinical evaluation can be used to identify movement patterns that fall outside of healthy pathology. By identifying such movement patterns, recommendations to change, modify, improve, or otherwise alter movement patterns can be made. In further embodiments, clinical evaluation can be used to identify one or more neurological disorders. The identifying neurological disorders can be based on muscle movements that include tremors, bradykinesia, muscle rigidity, walking or running gait, limb movement while swimming or dancing, and so on. In embodiments, analyzing muscle movements can be used to enhance diagnosis of one or more diseases or traumas such as Parkinson's disease, myotonic dystrophy, stroke, and so on.

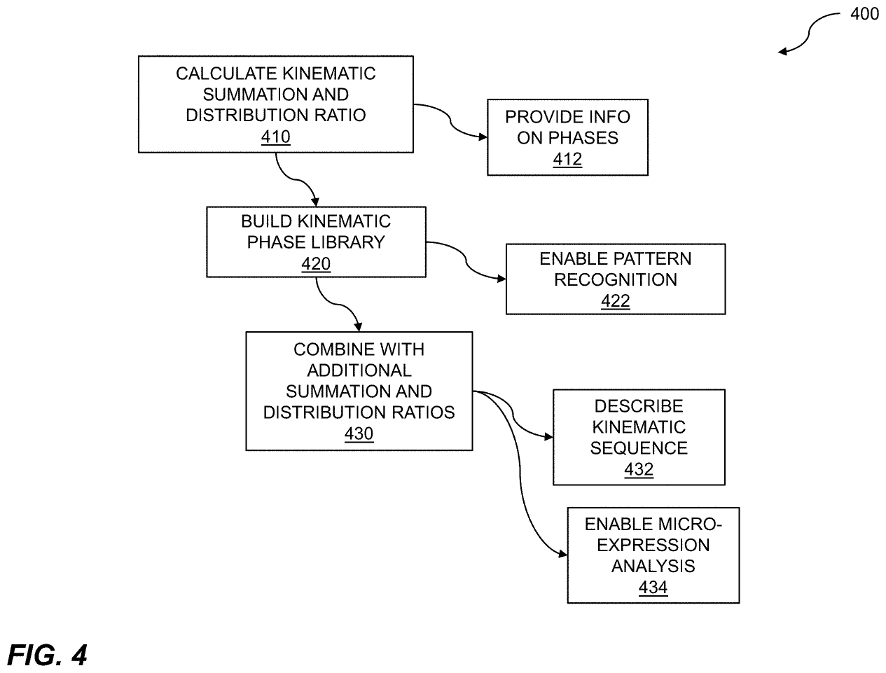

FIG. 4 is a flow diagram for calculating a kinematic summation and distribution ratio. A kinematic summation and distribution ratio can be calculated based on the microexpression of movement of a body part. The kinematic summation and distribution ratio can be used for body part biomarker and/or consistency pattern generation using motion analysis. Two or more sensors are attached to a body part of an individual, where the sensors enable collection of motion data of the body part. The sensors can include an inertial measurement unit, a muscle activation sensor, and so on. Sensor data is collected, where the data includes electrical information based on a microexpression of movement of the body part. The electrical information is analyzed to generate biomarker and/or consistency pattern information. The consistency pattern information is used for a clinical evaluation of the individual.

The flow 400 includes calculating a kinematic summation and distribution ratio 410 based on the microexpression of movement of the body part. The kinematic summation and distribution can be calculated using one or more processors. The calculating can be used to determine information such as position, rotation, movement, acceleration, and so on, related to the body part. In embodiments, the calculating provides information on kinematic phases 412. The kinematic phases can include phase patterns, where a kinematic phase pattern can refer to a cycle of movements of a body part, where such movements can be related to walking or running, raising and lowering arms, and so on. A phase can include a stance-phase. A stance-phase can include a double stance from walking. The double stance can include a heel strike, a mid-stance where the legs are vertical, a toe-off, and so on. The double stance can include a movement of a center of mass. In the case of walking, an individual's head will appear to rise at mid-stand and fall for heel-strike or toe-off. Another phase can include a swing-phase. In a swing-phase, a leg to be moved forward undergoes knee-flexion before being swung forward.

The flow 400 includes using the information on kinematic phases to build a kinematic phase library 420. A kinematic phase library can be used to collect, aggregate, store, etc., kinematic phase patterns. The kinematic phase patterns can result from analysis of the electrical information collected from the two or more sensors attached to the body part of the individual. The kinematic phase library can include data collected from other individuals. The data collected from the other individuals can represent populations of individuals, where the populations can include normal or healthy populations, injured populations, and so on. The data contained in the kinematic phase library can be compared to data from the individual to determine an injury, to measure progress of strengthening or healing, etc. The flow 400 can include using the kinematic phase library to enable pattern recognition 422 for information on kinematic phases obtained from the calculating. The pattern recognition can be used to recognize a kinematic phase; to measure a kinematic phase; to compare a kinematic phase to a “standard”, a “normal”, or a previous measurement obtained from the individual; and the like.

The flow 400 further includes combining the kinematic summation and distribution ratio with one or more additional kinematic summation and distribution ratios 430 for a segment of a related body part. The segment of a related body part can include muscles or joints adjacent to the body part, such as an elbow or a wrist adjacent to a shoulder to which the two or more sensors were attached. The segment of a related body part can include a symmetrical body part such as left or right shoulder, left or right hip, and so on. In embodiments, the combining can describe a kinematic sequence 432. A kinematic sequence can be used to describe a sequence of movements of a body part and how that sequence of movements can be used to transfer energy. A kinematic sequence can describe a jump or a launch from a starting stance for a sprint or an individual medley swimming event, a swing of a golf club or a tennis racquet, arm movement of a baseball pitcher, and so on. In embodiments, the combining can enable microexpression analysis 434 of the individual. The microexpression analysis can determine specific motions, paths of travel, velocity, acceleration, etc., related to the body part. The microexpression analysis can be applied to body part performance, therapies, and so on. In embodiments, the microexpression analysis of the individual can be used for athletic performance enhancement. The athletic performance enhancement can be used to optimize the swing of a golf club, tennis racquet, lacrosse stick, baseball or cricket bat, and the like. In further embodiments, the microexpression analysis of the individual can be used for medical treatment. The microexpression analysis can be used to design a rehabilitation therapy, to measure progress toward a rehabilitation goal, etc. In embodiments, microexpression analysis of the individual can be used for medical diagnostics. The diagnostics can include excessive motion of a joint, unbalanced movement of symmetrical joints, etc. In embodiments, the microexpression analysis can enable an objective measurement of scapular movement and dyskinesia. In some instances, the manner in which an individual moves a particular body part may increase a risk of injuring the body part. In embodiments, the microexpression analysis of the individual is used for injury risk analysis. An injury risk analysis can indicate that further tests, measurements, or diagnostics should be performed. In embodiments, the microexpression analysis of the individual is used for injury diagnostics.

Further to techniques that can calculate kinematic summation and a distribution ratio based on the microexpression of movement of a body part, other measurement techniques can be used. In embodiments, techniques based on electrical impedance myography can be explored. Electrical impedance myography (EIM) can be applied to a measurement of an intensity and a velocity of a muscle contraction event, where the measurement can include electrical impedance of the muscle or muscle group. EIM can be a noninvasive technique that can measure the electrical impedance characteristics. The electrical impedance characteristics can be used to determine health of a muscle or group of muscles, such as diagnosing a neuromuscular disease or other medical condition, assessing progression of the disease or condition, etc. The muscle health determination also can be useful for physical therapies, measuring the progress of healing, and so on.

The composition of a muscle or a group of muscles can be altered by the occurrence of disease, as can the microstructure of the muscle or group of muscles. By measuring changes in electrical impedance of the muscle or muscles using EIM, the occurrence of a disease such as a neuromuscular disease can be detected. The measurement of muscle impedance can be represented by a resistance-capacitance (RC) model, where the resistance component can be associated with cellular fluids within the muscles, and the reactance component can be associated with capacitive effects attributable to the cell membranes of the cells within the muscles. The cellular fluid can include extracellular fluid and intracellular fluid. The cell membranes can represent the capacitor dielectric separating the extracellular and intracellular fluids. Since disease can alter, sometimes significantly, the membranes of the cells, the cells can also undergo significant impedance changes. Thus, measuring the impedance of the muscle or muscle group over time can determine disease presence, disease progression, atrophy of muscle fibers, etc.

Impedance, such as electrical impedance associated with myography, is based on real components described as resistance, and imaginary components described as reactance. By applying a signal such as a sinusoidal signal to the surface of a muscle or muscle group, and by measuring the amount of time or time delay taken for the signal to pass through the muscle, a phase value can be calculated. By measuring resistance and reactance, and by calculating phase, a muscle disease may be identified. Electrical impedance myography can be impacted by physical characteristics of the patients for whom EIM is being performed. Physical characteristics of the patient can include thickness of the skin, the amount of fat under the skin (subcutaneous fat), and so on. By applying more than one sinusoidal test signal, where the sinusoidal test signals are based on different frequencies, the effects that skin and fat can have on impedance measurements can be reduced. Further, an amount of subcutaneous fat between the skin and the muscle may also be determined. In embodiments, at least one of the two or more sensors comprises an electromyogram sensor.

Other body part movement detection techniques include mechanomyography, which is sometimes called phonomyogram, acoustic myogram, or sound myogram. Mechanomyography relies on sensing muscle activation through resultant stimulation of adjacent media. For example, a muscle twitch can cause a movement of the skin above or near the muscle, which can then result in the movement of air molecules around the skin that can be detected as sound waves. Thus, in embodiments, at least one of the two or more sensors comprises a mechanomyogram sensor. Various steps in the flow 400 may be changed in order, repeated, omitted, or the like without departing from the disclosed concepts. Various embodiments of the flow 400 can be included in a computer program product embodied in a non-transitory computer readable medium that includes code executable by one or more processors.

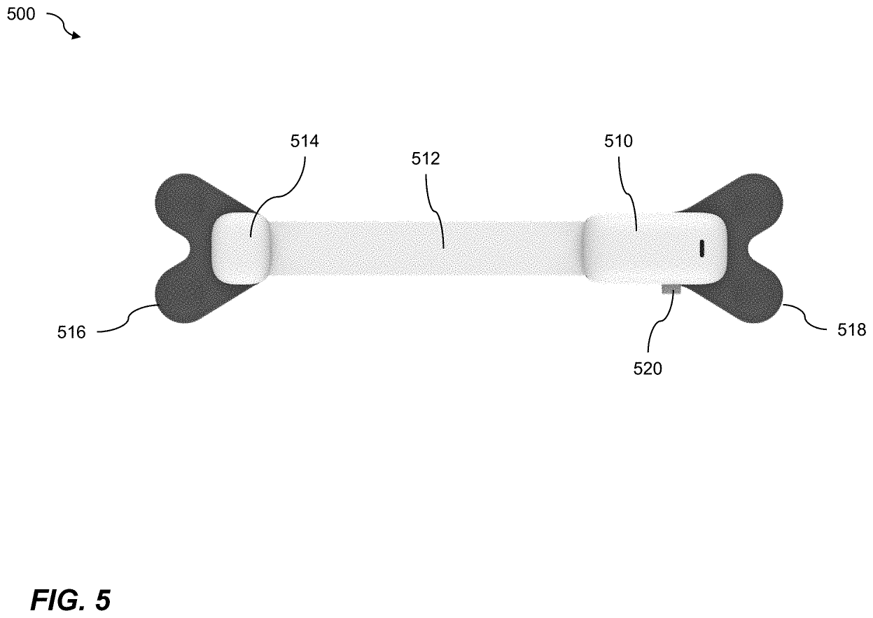

FIG. 5 shows sensor configuration. Discussed below and throughout, two or more sensors can be attached to a body part of an individual and can be used to collect motion data. The motion data can be analyzed for body part biomarker and/or consistency pattern generation. The sensors that can be attached can include stretch sensors, inertial measurement units (IMUs), sensors for detecting muscle activation, stretch sensors, and so on. An IMU can include one or more of an accelerometer, a gyroscope, and a magnetometer. Data is collected from the sensors, where the sensor data includes electrical information based on a microexpression of movement of the body part. Processors are used to analyze the electrical information in order to generate biomarker and/or consistency pattern information. The consistency pattern information can be augmented by analyzing additional data collected from the sensors. The augmented consistency pattern information can be used for a clinical evaluation for the individual.

A stretch sensor configuration 500 for attachment to a body part is shown. An IMU, a sensor for determining muscle activation, a stretch sensor, or other sensors can be used for body part biomarker information generation using motion analysis. The electrical characteristics of a sensor, such as an IMU, a muscle activation sensor, or a stretch sensor, change as the IMU or sensor moves, activates, or stretches, respectively. The electrical characteristics can include resistance, capacitance, inductance, reluctance, and so on. The muscle activations determined by the muscle activation sensor can correspond to movement of a body part to which the sensor is attached. Similarly, motion of an IMU can include acceleration, rotation, or position of the body part. A collector or sensor coupled to the muscle activation sensor collects changes in electrical characteristics based on motion which results from muscle activation of the body part. A communication unit provides information from the sensor or collector to a receiving unit. The stretch sensor configuration 500 can comprise an apparatus for attachment to tape on a body part. The sensor configuration can include an electrical component 510. The electrical component 510 can be coupled to a stretch sensor 512 and can collect data relating to changes in electrical characteristics of the stretch sensor 512. The electrical component 510 can include a power source that can provide power to electrical circuits and can drive the stretch sensor 512. The power source and circuitry provide other signals such as sinusoids or pulses at various frequencies, AC or DC voltages, etc., that may be required to operate the sensor. The electrical component can include an electrical characteristic calculation component. The electrical characteristic calculation component can be used to determine stretch, bulge, displacement, and other physical characteristics based on body part motion. The electrical component can include a Bluetooth™, Wi-Fi, Zigbee, or some other communication unit which can be used to send collected changes in electrical characteristics of the stretch sensor. While one stretch sensor is shown, other numbers of stretch sensors can be included in a sensor configuration. As stated throughout, additional sensors can be based on IMUs. The electrical component can be coupled to a button 520, switch, or other device for energizing or deenergizing the electrical component.

The stretch sensor 512 can include various materials which can be used to detect or measure stretch. In embodiments, the stretch sensor can include an electroactive polymer. The stretch sensors can be configured in a variety of arrangements such as a t-shape, an offset-t-shape, a w-shape, an x-shape, a spider-shape, and so on. The stretch sensor 512 can be coupled to an anchor 514 for the stretch sensor. The stretch sensor anchor can include hooks, and the anchor can be used to attach the stretch sensor to an anchor 516 and 518. The anchors 516 and 518 can include tape, fabric, wrap, and so on. When tape is used, the tape can be attached to the body part where the first stretch sensor can then be attached to the tape. In embodiments, the tape can include physical therapy tape. In other embodiments, the tape can include kinesiology therapeutic tape.

In other embodiments, the sensor configuration 500 can include a bend sensor. One or more bend sensors can be applied to a body part of an individual and can be used for body part motion analysis. The one or more bend sensors can be used to measure motion of the body part with one or more degrees of freedom. Various techniques can be used to implement a bend sensor such as configuring the bend sensor based on a compliant capacitive strain sensor. A compliant capacitive strain sensor can comprise a dielectric layer sandwiched between two conducting electrode layers. The dielectric layer and the electrode layers can be based on flexible materials, where the flexible materials can include polymers. The flexible materials such as the polymers can include natural rubber, silicone, acrylic, and so on. Since the polymers can typically be insulators, the electrodes of the bend sensor can be formed by introducing conducting particles into the polymers, where the conducting particles can include nickel, carbon black, and the like. In order for the capacitive strain sensor to be applied to the body part, one or more compliant capacitive strain sensors or other strain sensors can be applied to a material such as tape that can be applied to the body part, a fabric that can enwrap the body part, a garment that can be worn on the body part, and so on. In embodiments, at least one of the two or more sensors comprises a bending sensor.

The compliant capacitive strain sensor can measure strain based on the amount of displacement experienced by the strain sensor. The ability of a compliant capacitive strain sensor to measure strain can be limited by the amount of displacement that can be sustained by the strain sensor before the strain sensor is temporarily or permanently damaged. Excessive strain applied to the strain sensor can cause electrical parameters of the strain sensor, such as the resistance of the strain sensor, to change significantly. The significant change in resistance of the strain sensor can include an “open circuit” (high resistance) resulting from a damaged or destroyed strain sensor.

An application of a sensor, such as the configuration shown, to a body part (e.g., a shoulder) can be used to determine angle measurements for the shoulder. In embodiments, angle measurements can include sagittal plane flexion and extension. In addition to angle measurements for a given body part, muscle function assessment can also be performed. In embodiments, muscle function assessment can include displacement of muscle contraction that can occur during an activity. The activity can include normal physical activity such as yoga and strenuous physical activity such as swimming, rowing, rock climbing, and so on. Peak displacement of a muscle can be based on maximum contraction of key superficial muscle groups. A sensor can be attached to a targeted muscle group, over the location of greatest muscle mass displacement. In addition to peak muscle displacement for muscle function determination, an amount of time required to reach peak muscle contraction can be recorded. Other sensors can be applied to shoulder measurements. In embodiments, the inertial measurement unit (IMU) can be used to track acceleration and orientation of a body part such as a shoulder. Based on measurements collected from the IMU, intersegmental movement can provide information on movement patterns across anatomical joints. The information based on the intersegmental movement provides information on a fluidity of movement and a quality of motion. This information can provide side to side comparison of movement of the anatomical joints for healthy populations in contrast with injured populations.

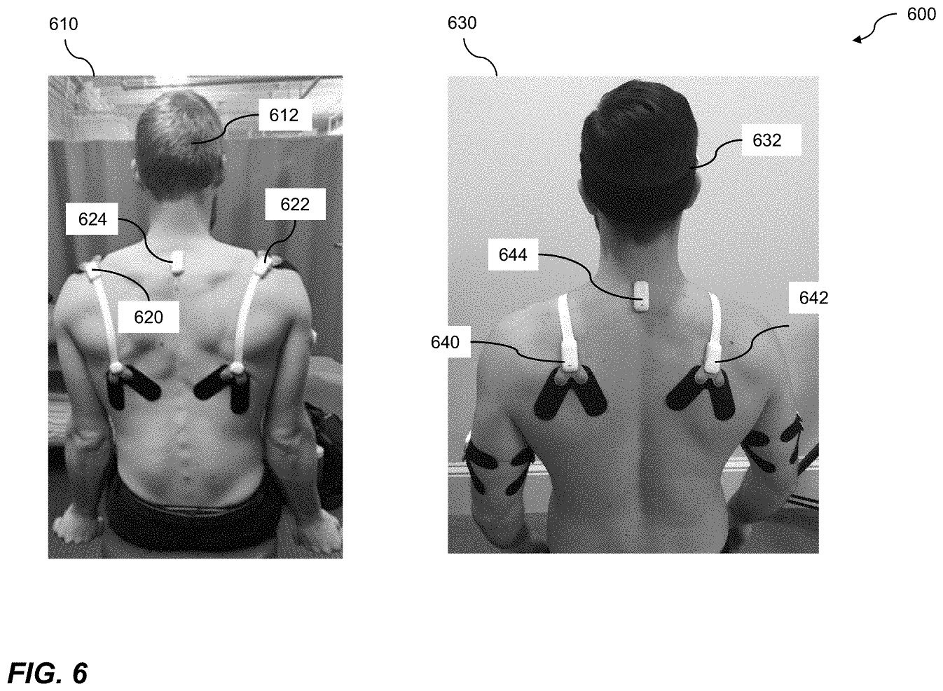

FIG. 6 illustrates sensor placement and alternative sensor placement 600. Sensors can be placed on a body part of an individual, where the sensors can include inertial measurement units (IMUs), sensors for determining muscle activation, stretch sensors, and so on. The sensors can enable collection of motion data of the body part. Data is collected from the sensors, where the data includes electrical information based on a microexpression of movement of the body part. The electrical information is analyzed to generate biomarker or consistency pattern information for the individual. The biomarker and/or consistency pattern information is used for a clinical evaluation of the individual. A placement 610 of sensors on an individual 612 is shown. The sensors, which can include two or more sensors, can be placed on a body part such as hips, knees, ankles, wrists, elbows, or shoulders, as shown. Further embodiments include attaching at least a third sensor to the body part. The third sensor can include an IMU, a muscle activation sensor, a stretch sensor, or some combination of sensors. In embodiments, the at least a third sensor can enable body part symmetry analysis. In the figure, three sensors are shown. A first sensor 620 can be attached to the left shoulder of the individual, a second sensor 622 can be attached to the right shoulder of the individual, and in embodiments, a third sensor 624 can be attached high and across the back of the individual. An alternative placement 630 of sensors is also shown, where the sensors are attached to an individual 632. The sensors in this alternate configuration, as in the first configuration, can include two or more sensors. The two or more sensors can be placed on a body part, a joint, a limb, etc. In the alternative configuration, sensors are placed on the left scapula and the right scapula. A third sensor is further attached to the body part, where the third sensor can enable body part symmetry analysis. In the figure, three sensors are shown. A first sensor 640 can be attached to the left scapula of the individual, a second 642 can be attached to the right scapula of the individual, and in embodiments, a third sensor 644 can be attached high and across the back of the individual for symmetry and other measurements.

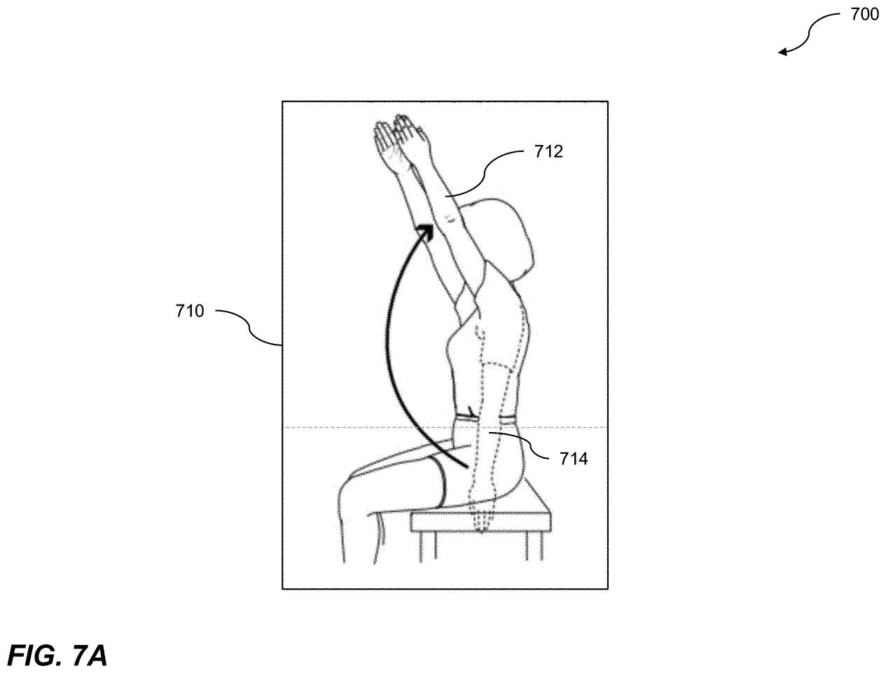

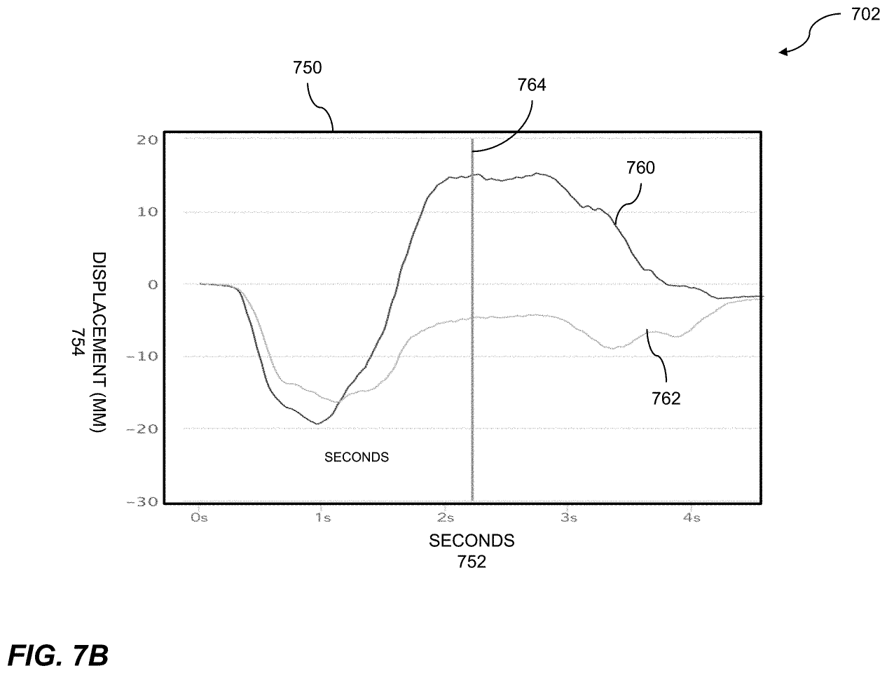

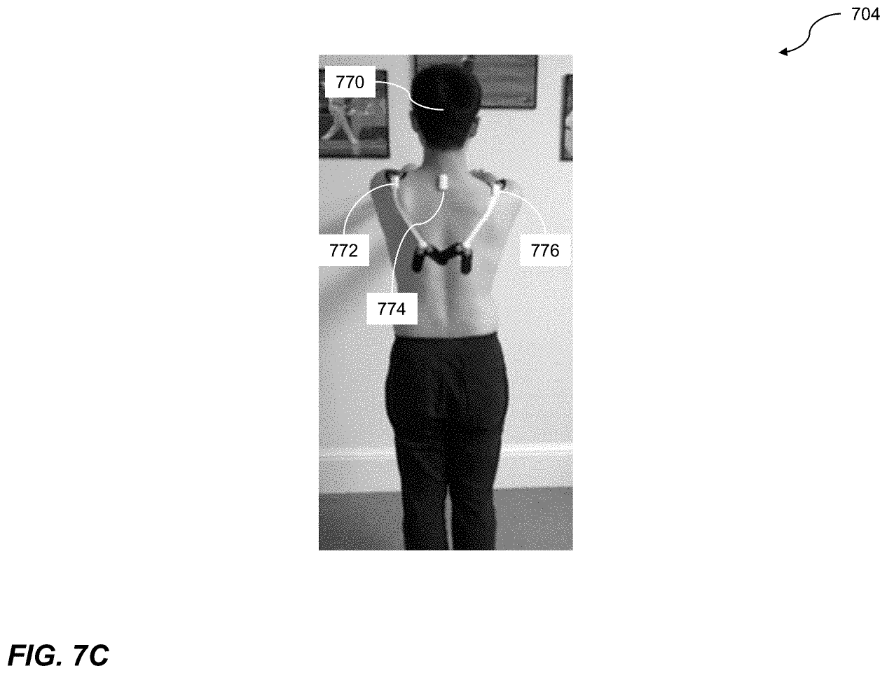

FIG. 7A shows shoulder motion. Two or more sensors such as inertial measurement units (IMUs), muscle activation sensors, stretch sensors, etc. can be used for body part biomarker and/or consistency pattern generation using motion analysis. The sensors are attached to a body part of an individual. The sensors enable collection of data of the body part, where the data can include motion data, activation data, and the like. Sensor data is collected, where the sensors provide electrical information based on a microexpression of movement of the body part during a movement performance protocol. Processors are used to analyze the electrical information to generate biomarker and/or consistency pattern information. Additional data can be subsequently collected and analyzed to provide additional information. The consistency pattern information and the additional consistency pattern information are used to generate further consistency pattern information. The consistency pattern is used for clinical evaluation for the individual.

FIG. 7A shows an example of shoulder motion 700. Two or more sensors such and IMUs and muscle activation sensors can be attached to left and right shoulders of a person 710, where the person can be a surgery patient, an injury patient, a test subject, and so on. The sensors attached to the shoulders of the patient can be used to test for and quantify a severity and a location of a loss of joint stability. In the figure, the patient can raise 712 or lower 714 her left and right arms together as the motion of her left and right shoulders is measured. Embodiments include attaching at least a third sensor to the body part, where in this example, the body part is the shoulder region of the individual. The third sensor can be used to enable body part symmetry analysis. The body part symmetry analysis can be used to determine that two body parts, such as left shoulder and right shoulder, are moving properly. In embodiments, the at least third sensor enables an objective measurement of scapular movement.

FIG. 7B shows data collected from shoulders 702. The data can be collected from a single individual, a group of individuals, and so on. The data that can be collected can include data related to a clinical evaluation or diagnosis, a therapy, and the like. The collected data can include electrical information from an IMU, a sensor determining muscle activation, a stretch sensor, and so on. Plot 750 can show the flex return of the left scapula 760 and the flex return of the right scapula 762. The flex return of the left scapula and the flex return of the right scapula can be measured while an individual is executing a movement, performing an action, etc. In embodiments, the movement can be based on a movement performance protocol. The plot 750 can include a time scale in seconds 752 and a displacement in millimeters 754. The plot 750 shows that the percent displacement of the left scapula and the percent displacement of the right scapula differ. The difference in percent displacement can be associated with a surgery, an injury, and so on. The motion of the body part can be measured as the patient performs an action such as a forward reach activity, an overhead reach activity, a side-to-side reach activity, a backwards reach activity, and the like. Similar arm reaching activities can be performed with the patient holding one or more weights. A weight can be held in each hand, the weight can be shared between the left hand and the right hand, etc. The vertical line 764 can denote a particular point in time during the reach activity or other activity. For this example, less displacement of the scapula is preferred to more displacement. An increase in displacement of a scapula can indicate impairment, injury, damage, etc.

FIG. 7C shows sensor position for data collection from the shoulders 704. Wearable sensors can be used to collect data relating to a body part of an individual. The body part can include a muscle, a joint, a limb, etc. The data that is collected can include motion data, where the motion data can be analyzed and used for body part biomarker and/or consistency pattern generation. The data that is collected can include electrical information from the one or more sensors such as an IMU, a muscle activation sensor, a stretch sensor, or one or such as optical sensors. The electrical information can be based on a microexpression of movement of the body part during a movement performance protocol. A movement performance protocol can include a reach activity. Positionings of sensors for data collection from an individual for shoulder motion are shown. Sensors can be applied to body parts such as shoulders of the individual 770. The sensors can be mounted as shown at the top of the left scapula 772, on a torso centerline 774, at the top of the right scapula 776, and so on. In embodiments, the sensors can be mounted at other positions on the scapulae, at a different point of the torso centerline or spine, on other joints or body parts, and so on. The body parts or locations can include individual body parts such as an arm, shoulder, hip, knee, leg, etc. The sensors can include IMUs, muscle activity or activation sensors, linear displacement or stretch sensors, and so on. The body parts or locations can include symmetrical locations such as left and right shoulder, elbow, hip, or knee; left and right arm or leg; and the like. The sensors which can be mounted can include single-type sensors such as IMU, muscle activity, or linear displacement sensors; or can include combination sensors that can comprise two or more types of sensors. In embodiments, the “combination” sensors can include IMU and muscle activation sensors.

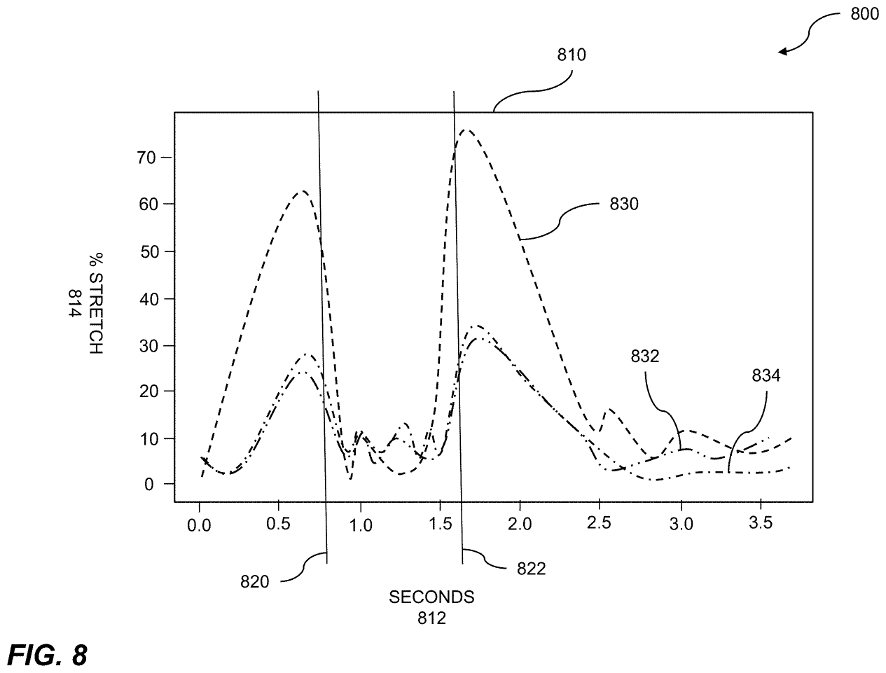

FIG. 8 illustrates a plot of jump data 800. Body part biomarker and/or consistency pattern generation uses motion analysis. Two or more sensors such as an inertial measurement unit (IMU) and a sensor determining muscle activation are attached to a body part, where the sensors can enable collection of motion data of a body part. Data is collected from the sensors, where the sensors provide electrical information. The electrical information is based on microexpression of body part movement during a movement performance protocol. The electrical information is analyzed to generate biomarker and/or consistency pattern information for the individual. A plot 810 of jump data is shown. The two or more sensors can be attached to joints such as a hips, knees, or ankles. The sensors can be attached to muscles such as quadriceps, calf (gastrocnemius and soleus) muscles, and so on. Data based on electrical information collected from stretch sensors or IMUs can be analyzed and plotted. The plot 810 can include a percentage stretch 814 versus time in seconds 812. The plot for the jump can show jump takeoff 820 and jump landing 822. The takeoff can correspond in time to a maximum compression of left 832 or right 834 quadriceps, and landing can similarly correspond in time to a maximum compression of left or right quadriceps. The plot for deflection of or force on left knee 830 or right knee can also be shown. Changes in electrical characteristics by a stretch sensor or an IMU can be rendered, along with an animation. The animation can include a human body, a body part of the human body, etc.

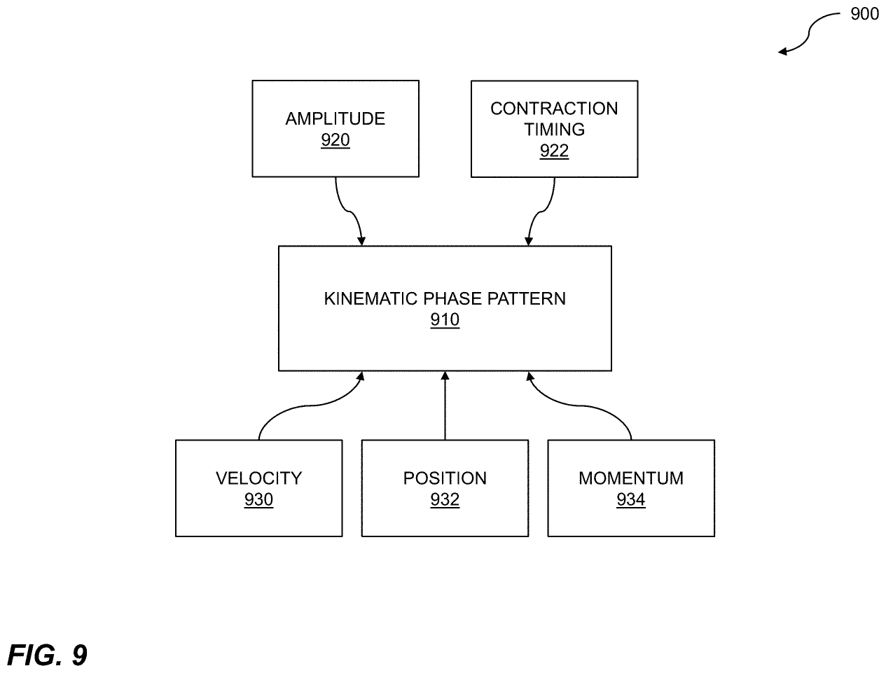

FIG. 9 shows a block diagram for a kinematic phase pattern from muscle data. Sensors, including two or more sensors attached to a body part of an individual, enable collection of motion data of the body part. The collected body part motion data enables body part biomarker and/or consistency pattern generation using motion analysis. The sensors include an inertial measurement unit (IMU) and a muscle activation sensor. The sensors provide electrical information based on microexpression of movement of the body part during a movement performance protocol. The electrical information is analyzed using processors to generate biomarker information which can be used for a clinical evaluation for the individual. The sensors that provide the electrical information can include an accelerometer, a gyroscope, or a magnetometer, a sensor for determining muscle activation, a stretch sensor, etc.

The block diagram 900 includes a kinematic phase pattern 910. A kinematic phase pattern can include one or more of angles, positions, accelerations, and velocities of segments of body parts and joints during the motions of the body parts and joints. A kinematic phase pattern can refer to a cycle of movements such as movements related to walking or running, raising and lowering arms, and so on. A phase can include a stance-phase. A stance-phase can include a double stance from walking. The double stance can include a heel strike, a mid-stance where the legs are vertical, a toe-off, and so on. The double stance can include a movement of a center of mass. In the case of walking, an individual's head will appear to rise at mid-stand and fall for heel-strike or toe-off. Another phase can include a swing-phase. In a swing-phase, a leg to be moved forward undergoes knee-flexion before being swung forward.

In order to form a kinematic phase pattern, various types of information can be collected. The types of information can be based on microexpressions of movement of the body part of the individual. In embodiments, the microexpression of movement of the body part includes muscle contraction amplitude 920 and muscle contraction timing 922. The muscle contraction amplitude and the muscle contraction timing can be collected from one or more sensors such as stretch sensors. In other embodiments, the microexpression of movement of the body part can include linear movements and rotational movements. Linear movements can include raising or lowering arms or legs, while rotational movements can include clockwise or anticlockwise rotations of shoulders, elbows, wrists, hips, knees, or ankles. In embodiments, the linear movements and the rotational movements each can comprise velocity 930, position 932, and momentum 934. The velocity, position, and momentum can be collected from one or more sensors such as inertial measurement units (IMUs). In embodiments, the velocity, position, and momentum each can include a magnitude and a time-dependent function. The velocity, position, and momentum of the body part change as the individual moves that body part. Various coordinate systems can be used to describe microexpressions of movement of the body part. In embodiments, the position can comprise a three-dimensional coordinate. Momentum can also be referenced using a variety of techniques. In embodiments, the momentum can include a center mass of a segment of a body part.

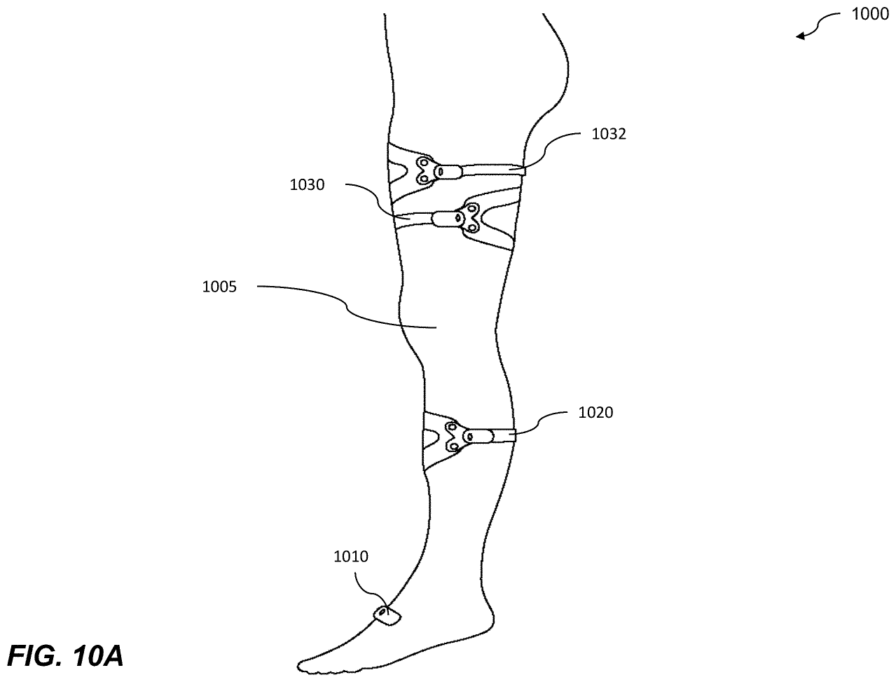

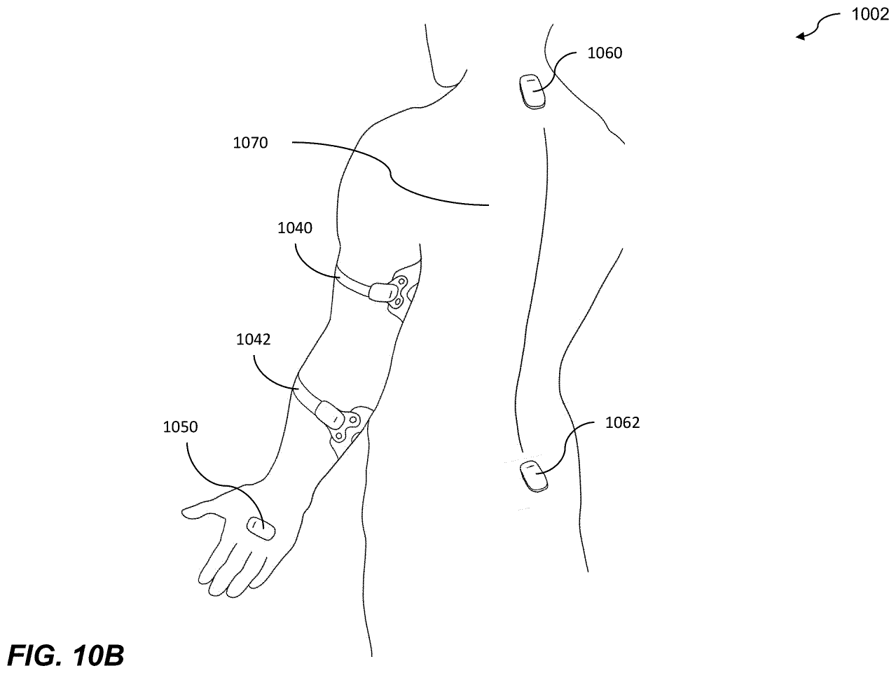

FIG. 10A shows example lower body sensor locations 1000. As discussed throughout, one or more wearable sensors can be used to collect data relating to a body part of an individual. The data that is collected can include electrical information from the one or more sensors. The body part can include a muscle, a joint, and so on. The collected electrical information can be analyzed to generate biomarker and/or consistency pattern information for an individual. The biomarker and/or consistency pattern information can be used for clinical evaluation for the individual. The lower human body 1005 to which sensors of various types can be mounted is shown. The sensors can include IMUs, muscle activity sensors, linear displacement or stretch sensors, and so on. The sensor can be mounted to the human body at various locations and for a variety of purposes. The body parts or locations can include individual body parts such as an arm, shoulder, hip, knee, leg, etc. The body parts or locations can include symmetric locations such as left and right shoulder, elbow, hip, or knee; left and right arm or leg; and the like. The sensors which can be mounted on the human body can include single-type sensors such as IMU, muscle activity, or linear displacement sensors; or can include combination sensors that can comprise two or more types of sensors. In embodiments, the “combination” sensors can include IMU and muscle activation sensors.

In embodiments, sensors can be applied to one leg or both legs. In the figure, sensors 1030 and 1032 are applied to the thigh of a leg, and sensor 1020 can be applied to the calf of the leg. Sensors 1020, 1030, and 1032 can be combination sensors that include both IMUs and muscle activation sensors. In addition, sensor 1010 can be applied to the top of the foot of the lower body 1005. Sensor 1010 can be only an IMU sensor, which can provide baseline lower body positioning data and the like.

FIG. 10B shows example upper body sensor locations 1002. Discussed throughout, one or more wearable sensors can be used to collect data relating to a body part of an individual. The data that is collected can include electrical information from the one or more sensors such as an IMU, a muscle activation sensor, a stretch sensor, and so on. The electrical information can be based on a microexpression of movement of the body part during a movement performance protocol. The body part can include a muscle, a joint, a limb, etc. The collected electrical information can be analyzed to generate a body part biomarker and/or consistency pattern. The body part biomarker and/or consistency pattern can be generated using the analyzing of the electrical information from the movement performance protocol. An upper human body 1070 to which sensors of various types can be mounted is shown. The sensors can include IMUs, muscle activity or activation sensors, linear displacement or stretch sensors, and so on. The sensor can be mounted to the human body at various locations and for a variety of purposes. The body parts or locations can include individual body parts such as an arm, shoulder, hip, knee, leg, etc. The body parts or locations can include symmetrical locations such as left and right shoulder, elbow, hip, or knee; left and right arm or leg; and the like. The sensors which can be mounted on the human body can include single-type sensors such as IMU, muscle activity, or linear displacement sensors; or can include combination sensors that can comprise two or more types of sensors. In embodiments, the “combination” sensors can include IMU and muscle activation sensors.

In embodiments, sensors can be mounted at specific locations on the upper human body such as at the neck 1060, at the small of the back 1062, and so on. Sensor 1060 and sensor 1062 can include IMUs. Sensors 1060, 1062, and others (not shown) can be particularly useful for detecting, calculating, or determining body motions. Other sensors can be applied to various body parts. In embodiments, sensors can be applied to one arm or both arms. In the figure, sensor 1040 is applied to an upper arm, and sensor 1042 is applied to the lower arm. The arm sensors can include IMUs and/or muscle activity sensors. The arm sensors can be the same types of sensors or may include different types of sensors, such as IMU sensor 1050 on the back of a hand, which can be an IMU sensor used to determine baseline hand/arm movement and positioning. When sensors are applied to both arms, movement, muscle displacement, etc., can be compared between the arms. Such comparisons are useful for detecting imbalances or asymmetries between muscles of the limbs, evaluating recovery from injury, etc.

In other embodiments, not shown, other numbers of wearable sensors may be applied to the various body parts. The wearable sensors can include muscle activation sensors, skeletal movement sensors, linear displacement sensors, inertial measurement unit sensors, and so on. The sensors can be applied to body parts in order to measure muscle or joint activity, displacement, deformation, etc. A sensor can be applied to the knee, for example. The sensor can be used to measure motion of the knee, for example, and can record a number of degrees of flexion of the knee as the person to whom the sensor is applied engages in various activities such as standing, walking, running, bicycling, dancing, swimming, and so on. Other sensors may be applied to the leg. A sensor can be applied to a quadriceps muscle. A sensor can be applied to a calf. The data collected or obtained from the sensors may be aggregated. Sensors can be applied to one leg or both legs, one arm or both arms, one scapula or both scapulae, etc. When sensors are applied to both legs, or other limbs, for example, the data collected from the sensors of the left limb and the sensors of the right limb can be analyzed for symmetry such as body posture symmetry. The data obtained from the sensors may also be used to quantify differences in muscle activity, joint movement, etc. The quantified difference can be correlated with changes in footwear, protective gear, clothing, and so on.

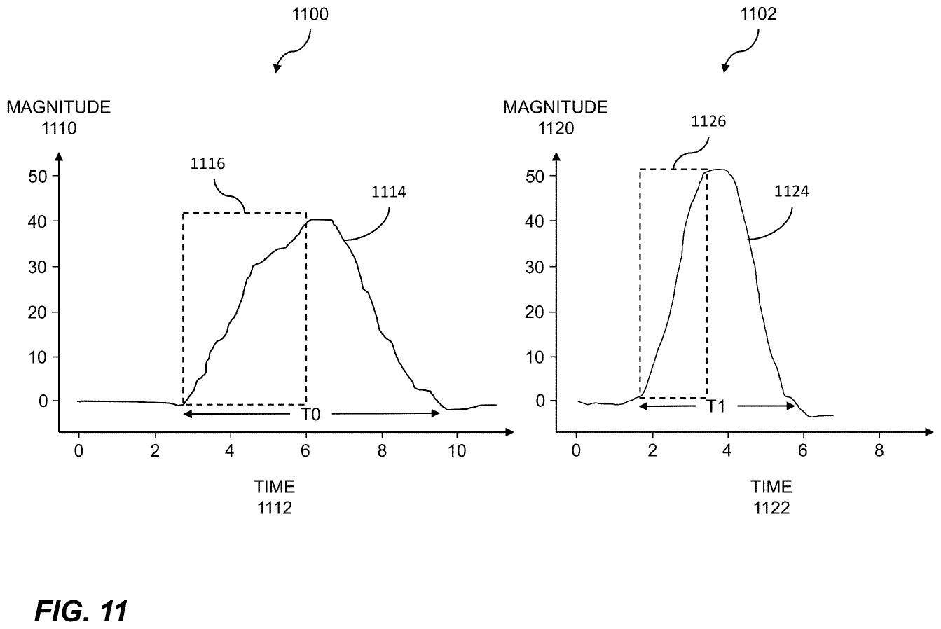

FIG. 11 illustrates neck range of motion differences. A normal range of motion of an individual's neck can be based on an individual being healthy, uninjured, physically fit, and so on. In contrast, the neck range of motion can be restricted, shaky, reduced, etc., due to injury, degeneration, surgery, and the like. The neck range of motion can be measured, tracked, and so on based on collecting data from sensors attached to the neck, shoulders, etc., of the individual. Measuring neck range of motions differences is enabled by body part biomarker and/or consistency pattern generation using motion analysis. Joint movement comparison enables body part biomarker and/or consistency pattern generation using motion analysis. Two or more sensors are attached to a body part of an individual, wherein the two or more sensors enable collection of motion data of the body part, wherein the two or more sensors include at least one inertial measurement unit (IMU) and at least one sensor determining muscle activation, and wherein the muscle activation comprises muscle deformation timing and muscle deformation displacement. Data from the two or more sensors is collected, wherein the two or more sensors provide electrical information based on a microexpression of movement of the body part during a movement performance protocol. The electrical information from the two or more sensors is analyzed using one or more processors. Biomarker information is generated for the individual, using the analyzing of the electrical information from the movement performance protocol.

Collected neck range of motion data is plotted for an injured neck 1100 and for a healthy neck 1102. While a motion of neck is described here, the motion can include motion of another body joint such as a shoulder, elbow, wrist, hip, knee, or ankle. In embodiments, the injury can include neck strain. The motion could also refer to motion of a muscle or muscle group. The data can be collected from sensors attached to an individual, obtained from a library of collected data, and so on. The data can include data collected before and after an injury, pre-operation and post-operation, etc. The data can include an individual's data compared to data obtained from a library of data, and the like. The data can include data collected over a period of time and can be used to measure healing, efficacy of a treatment plan, and so on. The collected neck range of motion data can be used to generate a biological marker or “biomarker” such as a range of motion biomarker. Additional biomarkers can be generated based on collected additional data such as the data collected over a period of time. The biomarkers, the additional biomarkers, etc. can be used for clinical evaluation of the individual. The clinical evaluation can be used to determine an injury, an extent or degree of the injury, and so on. In embodiments, the evaluation can enable classification of a degree of an injury. The classification of the degree of the injury can include qualitative classifications such as “injured” and “healed”. The classification can include an extent of the injury such as “mild” and “severe”. In embodiments, machine learning is employed to analyze the biomarkers, additional biomarkers, consistency pattern information, and so on.

A plot of neck range of motion is shown for an injured neck 1100. The plot can show a magnitude 1110 of motion versus time 1112. Sensor data can be collected from attached sensors, analyzed, and plotted 1114. The collected sensor data can be associated with a motion of the neck such as raising or lower a head (e.g., nodding “yes”), rotating the head to the left or right (e.g., nodding “no”), tilting the head to the left or right (e.g., tilting left ear toward left shoulder), and so on. The rising portion 1116 of the graph 1114 shows a rough or shaky transition from a starting point such as chin lowered to an end point such as chin raised. The transition can show that the individual experienced some pain during the transition, a restriction in the movement such as a “hitch”, an inability to attain a full or normal range of motion, etc. A period of time T0 during which the motion takes place can be longer than for motion of a healthy or uninjured neck. A plot of neck range of motion for a healthy neck 1102 is shown. The plot of the range of motion of the healthy neck can be associated with the individual, can be obtained from a library, and so on. The plot of the healthy neck range of motion can be based on collected sensor data. The collected sensor data can include sensor data collected prior to an injury, subsequent to treatment of an injury or postoperative therapy, and so on. The sensor data can be based on electrical information associated with the sensors, and can be plotted 1124 based on a magnitude 1120 versus time 1122. The rising portion 1126 of the graph 1124 shows a faster and smoother rise in comparison to the rise of the graph associated with the injured neck. Further, a period of time T1 associated with the motion of the healthy neck is smaller in comparison to the time associated with the injured neck. The smoother motion and the shorter amount of time during which the motion takes place can indicate that an injury sustained by the individual has healed, that a treatment was successful, and so on.

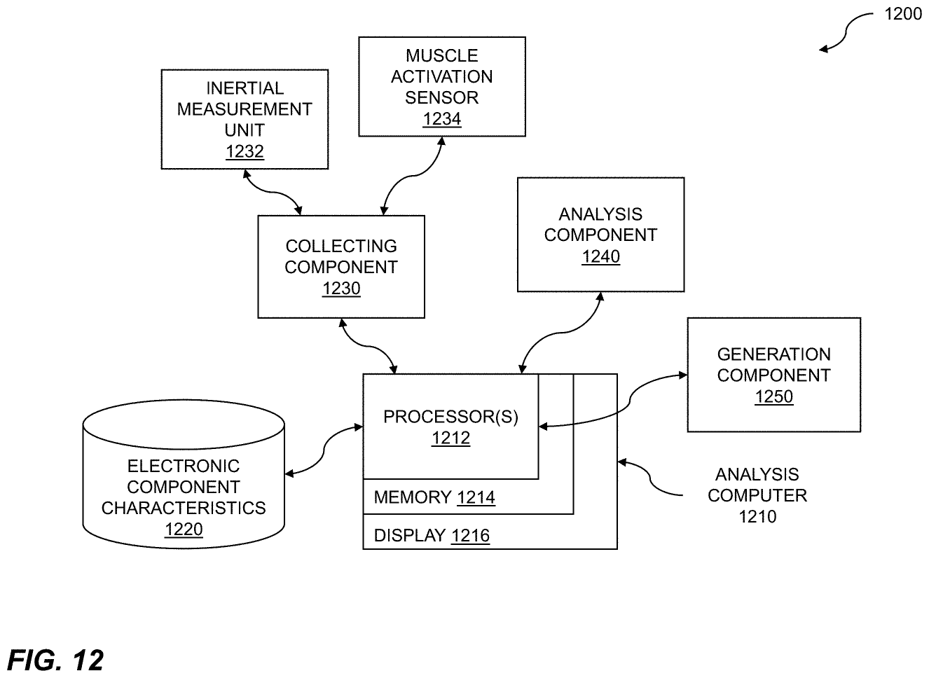

FIG. 12 is a system diagram for body part biomarker and/or consistency pattern generation using motion analysis. Motion analysis can describe motion of a body part including a joint such as a shoulder, elbow, hip, knee, ankle, wrist; a limb such as an arm or leg; and so on. The motion analysis can be used to determine extent of an injury, progress toward recovery, and the like. Sensors, including body-attachable sensors, can be used to analyze motion of a body part. Sensors can be applied to a body part of an individual, where the application can be accomplished using tape or straps using hooks, suction cups, wraps, and so on. The sensors can include inertial measurement units (IMUs), muscle activation sensors, stretch sensors, etc. IMUs can include an accelerometer, a gyroscope, a magnetometer, etc. Information can be collected from the IMU or muscle activation sensors, where the information can include changes in electrical characteristics of the IMU or muscle activation sensors. The electrical characteristics can change based on the sensor stretching, accelerating, moving, rotating, and the like. The electrical characteristics can include electrical information, where the electrical information can be based on microexpressions of movement of the body part during a movement performance protocol. Processors can be used to analyze the electrical information collected from the sensors. The collected electrical information can be used to generate biomarker and/or consistency pattern information for an individual. The consistency pattern information can be used to measure improvements, changes, or deterioration of movement of a muscle or joint; to identify movement patterns that fall outside of healthy pathology; to determine clinical evaluations and to recommend treatment plans; to determine malingering; etc. In embodiments, the generated consistency pattern can be rendered as an animation of the body part. The data relating to the generated consistency pattern for the body part can be used for body part treatment including medical techniques, physical therapy, occupational therapy, athletic training, strengthening, flexibility, endurance, conditioning, or rehabilitation therapy treatment.

The system 1200 can include an analysis computer 1210. The analysis computer can include one or more processors used to analyze electrical information from two or more sensors. The analysis performed by the analysis computer can generate a body part biomarker information using motion analysis. The analysis computer 1210 can comprise one or more processors 1212, a memory 1214 coupled to the one or more processors 1212, and a display 1216. The display 1216 can be configured and disposed to present collected data, analysis, intermediate analysis steps, instructions, algorithms, or heuristics, and so on. The system 1200 can comprise a computer system for motion analysis comprising: a memory which stores instructions; one or more processors coupled to the memory wherein the one or more processors, when executing the instructions which are stored, are configured to: attach two or more sensors to a body part of an individual, wherein the two or more sensors enable collection of motion data of the body part, wherein the two or more sensors include at least one inertial measurement unit (IMU) and at least one sensor determining muscle activation, and wherein the muscle activation comprises muscle deformation timing and muscle deformation displacement; collect data from the two or more sensors, wherein the two or more sensors provide electrical information based on a microexpression of movement of the body part during a movement performance protocol; analyze the electrical information from the two or more sensors; and generate biomarker information for the individual, using the analyzing of the electrical information from the movement performance protocol. The muscle deformation can include volumetric changes of the muscle.

The system 1200 can include an electronic component characteristics component 1220. The electronic component characteristics can include a library of lookup tables, resistance characteristics, capacitance characteristics, functions, algorithms, routines, and so on, that can be used for the analysis of the electrical information collected from the one or more sensors. In a usage example, the electrical component characteristics can include a lookup table that enables mapping of an electrical signal from a muscle activation sensor to millimeters of motion of the body part. The system 1200 can include a collecting component 1230. The collecting component can act as an interface between one or more sensors and the analysis computer 1210. The collecting component can collect data based on electrical signals, where the electrical signals can be generated by an inertial measurement unit 1232, a muscle activation sensor 1234, a stretch sensor (not shown), etc. The collecting component can include resistance and/or capacitance measuring hardware, and can include hardware for measuring current, voltage, resistance, capacitance, impedance, and/or inductance.

The system 1200 can include an analysis component 1240. The analysis component can analyze the data based on electrical information that is collected from various sensors such as inertial measurement units, muscle activation sensors, stretch sensors, and so on. The analysis can be performed on electrical signals related to acceleration, rotational motion, magnetic field, activation, stretch, and the like. The system 1200 can include a generation component 1250. The generation component 1250 can include hardware for generating direct current and/or alternating current signals used for obtaining resistance and/or capacitance measurements. Typically, the current values are low (e.g., microamperes) and in embodiments, the frequency range includes signals from about 100 hertz to about 1 megahertz. The generating component can generate consistency pattern information for the individual. In embodiments, the consistency pattern can be used for a clinical evaluation for the individual, designing therapies such as physical therapy for recovery by the individual from injury or surgery, and the like.

The system 1200 can include a computer program product embodied in a non-transitory computer readable medium for motion analysis, the computer program product comprising code which causes one or more processors to perform operations of: attaching two or more sensors to a body part of an individual, wherein the two or more sensors enable collection of motion data of the body part, wherein the two or more sensors include at least one inertial measurement unit (IMU) and at least one sensor determining muscle activation, and wherein the muscle activation comprises muscle deformation timing and muscle deformation displacement; collecting data from the two or more sensors, wherein the two or more sensors provide electrical information based on a microexpression of movement of the body part during a movement performance protocol; analyzing, using one or more processors, the electrical information from the two or more sensors; and generating biomarker information for the individual, using the analyzing of the electrical information from the movement performance protocol.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A processor-implemented method for motion analysis comprising:
attaching two or more sensors to a body part of an individual, wherein the two or more sensors enable collection of motion data of the body part, wherein the two or more sensors include at least one inertial measurement unit (IMU) and at least one sensor determining muscle activation, and wherein the muscle activation comprises muscle deformation timing and muscle deformation displacement;
collecting data from the two or more sensors, wherein the two or more sensors provide electrical information based on a microexpression of movement of the body part during a movement performance protocol;
analyzing, using one or more processors, the electrical information from the two or more sensors; and
generating biomarker information for the individual, using the analyzing of the electrical information from the movement performance protocol.

2. The method of claim 1 further comprising collecting additional data from a subsequent attaching of two or more sensors to the body part of the individual.

3. The method of claim 2 further comprising analyzing the additional data to provide additional biomarker information.

4. The method of claim 3 further comprising generating a biomarker using the biomarker information and the additional biomarker information.

5. The method of claim 4 further comprising using the biomarker for a clinical evaluation for the individual.

6. The method of claim 5 wherein the clinical evaluation enables determination of malingering.

7. The method of claim 5 wherein the clinical evaluation enables classification of a degree of an injury.

8. The method of claim 7 wherein the injury is a neck strain.

9. (canceled)

10. The method of claim 5 wherein the clinical evaluation is monitored over time to produce a healing trajectory.

11. The method of claim 10 wherein the healing trajectory is compared to a library of healing trajectories.

12. The method of claim 10 wherein the healing trajectory is used to verify an extent of an injury.

13. The method of claim 5 wherein the clinical evaluation is used to identify movement patterns that fall outside of healthy pathology.

14. The method of claim 1 wherein the movement performance protocol includes task performance.

15. The method of claim 14 further comprising analyzing the task performance to identify movement dysfunction.

16. The method of claim 15 wherein the movement dysfunction that is identified enables determination of typical injury impairment.

17-19. (canceled)

20. The method of claim 1 wherein the two or more sensors capture two or more modalities of body part motion.

21. The method of claim 1 further comprising scoring mobility of the individual, based on the biomarker information.

22. (canceled)

23. The method of claim 1 wherein the electrical information that is provided comprises surface mechanomyography information.

24. The method of claim 1 further comprising using the biomarker information to identify a movement consistency pattern.

25. The method of claim 24 wherein the movement consistency pattern is used to detect an injury of the individual.

26. The method of claim 25 further comprising categorizing the injury of the individual based on the movement consistency pattern.

27. The method of claim 1 wherein the muscle deformation includes volumetric changes of the muscle.

28. The method of claim 1 wherein the microexpression of movement of the body part includes hold fatigue.

29. The method of claim 1 wherein the biomarker information for the individual comprises a consistency pattern.

30. A computer program product embodied in a non-transitory computer readable medium for motion analysis, the computer program product comprising code which causes one or more processors to perform operations of:
attaching two or more sensors to a body part of an individual, wherein the two or more sensors enable collection of motion data of the body part, wherein the two or more sensors include at least one inertial measurement unit (IMU) and at least one sensor determining muscle activation, and wherein the muscle activation comprises muscle deformation timing and muscle deformation displacement;
collecting data from the two or more sensors, wherein the two or more sensors provide electrical information based on a microexpression of movement of the body part during a movement performance protocol;
analyzing, using one or more processors, the electrical information from the two or more sensors; and
generating biomarker information for the individual, using the analyzing of the electrical information from the movement performance protocol.

31. A computer system for motion analysis comprising:
a memory which stores instructions;
one or more processors coupled to the memory wherein the one or more processors, when executing the instructions which are stored, are configured to:
attach two or more sensors to a body part of an individual, wherein the two or more sensors enable collection of motion data of the body part, wherein the two or more sensors include at least one inertial measurement unit (IMU) and at least one sensor determining muscle activation, and wherein the muscle activation comprises muscle deformation timing and muscle deformation displacement;
collect data from the two or more sensors, wherein the two or more sensors provide electrical information based on a microexpression of movement of the body part during a movement performance protocol;
analyze the electrical information from the two or more sensors; and
generate biomarker information for the individual, using the analyzing of the electrical information from the movement performance protocol.
